SEQUENCE LISTING (23 sequences)

SEQ ID NO: 1 - DNA, 22 bp, Artificial Sequence
  synthetic construct
cgcagtagtttgaaagtaaccc

SEQ ID NO: 2 - DNA, 22 bp, Artificial Sequence
  synthetic construct
caaacagattaataccctaagc

SEQ ID NO: 3 - DNA, 16 bp, Artificial Sequence
  synthetic construct
ccgtaggtggcatcgc

SEQ ID NO: 4 - DNA, 22 bp, Artificial Sequence
  synthetic construct
atctyaatttrggkcgygaaga

SEQ ID NO: 5 - DNA, 21 bp, Artificial Sequence
  synthetic construct
cacrtcmgcgtcraagaaata

SEQ ID NO: 6 - DNA, 21 bp, Artificial Sequence
  synthetic construct
ggtggaccttatggtgagaac

SEQ ID NO: 7 - DNA, 23 bp, Artificial Sequence
  synthetic construct
cacgtattggtagtgtggtcata

SEQ ID NO: 8 - DNA, 21 bp, Artificial Sequence
  synthetic construct
ttggttccgctagttcagaag

SEQ ID NO: 9 - DNA, 23 bp, Artificial Sequence
  synthetic construct
ggatattgggtgttaagttcgtc

SEQ ID NO: 10 - DNA, 24 bp, Artificial Sequence
  synthetic construct
caaaccctacatcatcgtcaaatc

SEQ ID NO: 11 - DNA, 25 bp, Artificial Sequence
  synthetic construct
ctaaggtttccgtctatccctaatc

SEQ ID NO: 12 - DNA, 22 bp, Artificial Sequence
  synthetic construct
cgatgacggttatagttggagg

SEQ ID NO: 13 - DNA, 20 bp, Artificial Sequence
  synthetic construct
tcgtctgttctctgcacttg

SEQ ID NO: 14 - DNA, 22 bp, Artificial Sequence
  synthetic construct
gagtggaaggattatctcagcc

SEQ ID NO: 15 - DNA, 21 bp, Artificial Sequence
  synthetic construct
acctcttgacaaccgtttcag

SEQ ID NO: 16 - DNA, 21 bp, Artificial Sequence
  synthetic construct
gtctcatcctcattaccctcg

SEQ ID NO: 17 - DNA, 18 bp, Artificial Sequence
  synthetic construct
cggagcgactggacattg

SEQ ID NO: 18 - DNA, 21 bp, Artificial Sequence
  synthetic construct
cccagttctcttaggcacaac

SEQ ID NO: 19 - DNA, 21 bp, Artificial Sequence
  synthetic construct
gccctcaaatatgtccctacg

SEQ ID NO: 20 - DNA, 20 bp, Artificial Sequence
  synthetic construct
gatggttttgcagatgcgag

SEQ ID NO: 21 - DNA, 21 bp, Artificial Sequence
  synthetic construct
tcctctggttccctgtagaag

SEQ ID NO: 22 - DNA, 21 bp, Artificial Sequence
  synthetic construct
gctttcagcctcttcgacaag

SEQ ID NO: 23 - DNA, 21 bp, Artificial Sequence
  synthetic construct
ccatcagcatccacctcattg

(19) United States
(12) Patent Application Publication
(10) Pub. No.: US 2019/0169624 A1
(43) Pub. Date: Jun. 6, 2019

(54) LOW INOCULUM, LONG CO-CULTURE AGROBACTERIUM-MEDIATED TRANSFORMATION OF PLANTS

(71) Applicant: OHIO STATE INNOVATION FOUNDATION, Columbus, OH (US)

(72) Inventors: John James FINER, Wooster, OH (US); Zhifen ZHANG, Tifton, GA (US)

(21) Appl. No.: 16/071,420

(22) PCT Filed: Jan. 19, 2017
(86) PCT No.: PCT/US2017/014104

§ 371 Date: Jul. 19, 2018

Related U.S. Application Data
(60) Provisional application No. 62/280,676, filed on Jan. 19, 2016.

(51) Int. Cl. C12N 15/82

(52) U.S. Cl. CPC C12N 15/8205; C12N 2310/20

(57) ABSTRACT

Disclosed herein is a method for producing genetically modified plants comprising inoculating a plant, plant cell, plant seed, or plant tissue with an Agrobacterium containing a nucleic acid of interest at an inoculum density of about 10-10,000 CFU/ml; and culturing the Agrobacterium-inoculated plant, plant cell, plant seed, or plant tissue for a period of from 7 days to 28 days, wherein said culturing results in a genetically modified plant, plant cell, plant seed, or plant tissue. Also disclosed are genetically modified plants, plant cells, plant seeds, or plant tissue based on the methods disclosed herein, as well as their progeny, and propagation material related thereto.

### CROSS-REFERENCE TO RELATED APPLICATIONS

This application is a national phase application of PCT US2017/014104, filed Jan. 19, 2017, which claims benefit of U.S. Provisional Application No. 62/280,676, filed Jan. 19, 2016, which is hereby incorporated herein by reference in its entirety.

### REFERENCE TO SEQUENCE LISTING

The Sequence Listing submitted with PCT Application US2017/014104 (of which the present application is a national phase application), filed Mar. 13, 2017, as a text file named “10336-211WO1_Invitation_ST25.TXT”, created on Mar. 13, 2017, and having a size of 5 KB, is hereby incorporated by reference pursuant to 37 C.F.R. § 1.52(e)(5).

### BACKGROUND

Since the first reports of using Agrobacterium tumefaciens to introduce genes into plant cells (Bevan et al. 1983; Fraley et al. 1983; Herrera-Estrella et al. 1983), Agrobacterium-mediated transformation has become the method of choice for gene introduction in most plant species (Fillatti et al. 1987; Bidney et al. 1992; Perl et al. 1996; Trick and Finer 1997; Bond et al. 1998; Clough and Bent 1998; Zhao et al. 2002; Cheng et al. 2003, 2004). With a more thorough understanding of how Agrobacterium delivers its transfer DNA (T-DNA) into plant cells and integrates it into the plant genome (Gelvin 2003, 2012), Agrobacterium-mediated plant transformation has been continuously improved by optimizing conditions for virulence gene induction (Godwin et al. 1991; Alt-Mörbe et al. 1989), developing high-virulence bacterial strains (Hood et al. 1993; Hansen et al. 1994), identifying crop-enhanced strains (Benzle et al. 2015), adopting efficient inoculation methods (Bidney et al. 1992; Trick and Finer 1997), and reducing plant defense responses (Perl et al. 1996; Olhoft and Somers 2001). Although different protocols have been developed for transformation of many plant species, plant tissues are always inoculated with millions of bacteria (Fillatti et al. 1987; Hiei et al. 1994; Bond et al. 1998; Zhao et al. 2002), usually as a suspension but occasionally as a bacterial colony. This approach is likely based on the predominant conception that the highest transformation rates result from the use of large numbers of bacteria to infect a large number of plant cells (Cheng et al. 2004).

When plant tissues are inoculated with A. tumefaciens, the presence of this plant pathogen can be detected by the plant defense system, inducing responses that may limit transformation and regeneration from transformed cells. Recognition of the pathogen-associated molecular pattern (PAMP), EF-Tu, from A. tumefaciens, by a plant kinase receptor (EFR) in Arabidopsis thaliana, reduced transformation by A. tumefaciens through PAMP-triggered immunity (PTI) responses (Zipfel et al. 2006). Additionally, induction of several plant defense genes was observed following inoculation of A. tumefaciens onto either A. thaliana cell cultures (Ditt et al. 2006), A. thaliana inflorescence stalks (Lee et al. 2009), tobacco cell cultures (Nicotiana tabacum, Veena et al. 2003), or wheat (Triticum aestivum) embryogenic calli (Zhou et al. 2013). If pathogen challenges continued, the intensity of defense responses further increased, leading to programmed cell death (PCD) or the hypersensitive response (HR) (Coll et al. 2011; Jones and Dangl 2006). Undesirable tissue browning, observed during Agrobacterium-mediated plant transformation, is commonly associated with HR (Perl et al. 1996; Hansen 2000; Olhoft and Somers 2001).

Considering that plant defense responses triggered by A. tumefaciens can reduce plant transformation efficiency, a reduction of plant defense activation can improve transformation rates. The use of antioxidants to suppress the oxidative burst, a common event at the early stage of HR (Lamb and Dixon 1997), has led to improvements in transformation of maize (Zea mays, Frame et al. 2002), soybean (Glycine max, Olhoft and Somers 2001), and grape (Vitis vinifera, Perl et al. 1996). Increases in transformation efficiency were also observed in the efr mutant of A. thaliana that lost the ability to recognize EF-Tu (Zipfel et al. 2006). The activation of plant defenses could depend on the inoculum density, as a threshold inoculum density of Pseudomonas fluorescens was required for the induction of systemic resistance in radish (Raphanus sativus, Leeman et al. 1995; Raaijmakers et al. 1995). Since induction of plant defense genes could result following inoculation of plant tissues with a high number of infecting cells, the use of low-density inoculum may enable A. tumefaciens to evade the host detection by not activating plant defense responses. This may be the likely scenario in field infestations, where low numbers of this bacterium found in the soil (Benzle et al. 2015) infect susceptible plants at wound sites.

Co-culture is critical for plant transformation because it is the period when A. tumefaciens cells interact with host cells and deliver the processed T-DNA into the targeted cells. Modification of co-culture conditions through medium modification or plant tissue preparation has been explored to increase transformation efficiency (Santarém et al. 1998; Olhoft and Somers 2001; Cheng et al. 2003). Regardless of the modifications, co-culture periods for most transformation protocols were limited to 2-3 days (Bidney et al. 1992; Perl et al. 1996; Trick and Finer 1997; Olhoft and Somers 2001; Cheng et al. 2003). Although an increase in the number of transformed cells was observed with 5-6 days co-culture periods in citrange (Citrus sinensis×Poncirus trifoliata, Cervera et al. 1998), rice (Oryza sativa, Rashid et al. 1996), and sunflower (Helianthus annuus, Sujatha et al. 2012), extensions of co-culture periods were not adopted due to severe bacterial overgrowth that suppressed plant regeneration. To prevent A. tumefaciens overgrowth during co-culture, approaches that could slow bacterial growth have been evaluated, such as placement of plant tissues on filter paper (Ozawa 2009), addition of silver nitrate to media (Zhao et al. 2002), and desiccation of explants (Cheng et al. 2003). With most of these approaches, the co-culture time remained less than 4 d, suggesting that slowing bacterial growth could not completely preclude unwanted damages to plant tissues associated with long co-culture. Although a 7-d co-culture was used to improve transformation in rubber tree (Hevea brasiliensis), a low co-culture temperature was used, which slowed the growth of the bacteria, (Blanc et al. 2006).

What is needed in the art are methods to significantly improve transformation rates by increasing the exposure time of Agrobacterium to the target tissue by using a combination of low-density inoculum of Agrobacterium with a long co-culture period.

### SUMMARY

Disclosed herein is a method for producing genetically modified plants comprising inoculating a plant, plant cell, plant seed, or plant tissue with an Agrobacterium containing a nucleic acid of interest at an inoculum density of about 10-10,000 CFU/ml; and culturing the Agrobacterium-inoculated plant, plant cell, plant seed, or plant tissue for a period of from 7 days to 28 days, wherein said culturing results in a genetically modified plant, plant cell, plant seed, or plant tissue. Also disclosed are genetically modified plants, plant cells, plant seeds, or plant tissue based on the methods disclosed herein, as well as their progeny, and propagation material related thereto.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-D show transformation using high inoculum and 3-day co-culture followed by hygromycin selection. FIG. 1A shows the cut side of a sunflower cotyledon at the top, 8 days after inoculation. FIG. 1B is an explant showing GFP in the cotyledon tissue with induced shoots on the adaxial side having no GFP expression 16 days after inoculation. FIGS. 1C and 1D show the cut side in contact with the medium 16 days after inoculation, with a single GFP shoot (arrowhead) and most GFP-expressing cells at the cut side; FIG. 1C is under GFP excitation conditions (GFP-2 filter); FIG. 1D is under brightfield (no filter). Bar=1 mm.

FIG. 2 shows GFP-expressing shoots observed on cotyledon explants. The use of low inoculum suspensions (approximately 6×10² CFU mL⁻¹) with 15-day-long co-culture resulted in the production of transformed cells at the shoot-forming regions with much higher efficiency, leading to a significantly higher number of GFP shoots than obtained using high inoculum. The high inoculum (OD600≈0.55) contained about 6×10⁸ CFU mL⁻¹, while its 10⁻², 10⁻⁴, 10⁻⁶, 10⁻⁸, and 10⁻¹⁰ dilutions contained about 6×10⁶, 6×10⁴, 6×10², 6, and 6×10⁻² CFU mL⁻¹, respectively. The use of a low inoculum suspension led to a significant increase in the production of GFP shoots after a 15-day-long co-culture. Bar=1 mm.

FIGS. 3A and 3B show the transformation efficiency of cotyledon explants. The high inoculum (OD600≈0.55) contained about 6×10⁸ CFU mL⁻¹, while its 10⁻², 10⁻⁴, 10⁻⁶, 10⁻⁸, and 10⁻¹⁰ dilutions contained about 6×10⁶, 6×10⁴, 6×10², 6, and 6×10⁻² CFU mL⁻¹, respectively. FIG. 3A shows mean percentages of explants having GFP shoots. FIG. 3B shows means of GFP shoots per explant. Bars represent standard error, with different letters indicating significant difference based on Duncan's multiple range test (p<0.05). Values are mean±standard error.

FIG. 4 shows shoot organogenesis of cotyledon explants after inoculation with Agrobacterium suspensions. The high inoculum (OD600≈0.55) contained about 6×10⁸ CFU mL⁻¹, while its 10⁻², 10⁻⁴, 10⁻⁶, 10⁻⁸, and 10⁻¹⁰ dilutions contained about 6×10⁶, 6×10⁴, 6×10², 6, and 6×10⁻² CFU mL⁻¹, respectively. Inoculation was followed by 15 day co-culture. Data represent the mean percentage of cotyledon explants forming shoots (white) and the mean of shoots per explant (gray) after 15 days of culture. Bars represent standard error, with different letters within each group (white or gray, no mark or prime respectively) indicating significant difference based on Duncan's multiple range test (p<0.05). Values are mean±standard error.

FIG. 5 shows the change in the percentage of explants with detectable Agrobacterium during 15-day co-culture after inoculation of explants with either low inoculum (low−6×10² CFU mL⁻¹) or high inoculum (high−6×10⁸ CFU mL⁻¹). Values are mean±standard error.

FIGS. 6A-F show relative expression levels of HaPR1, HaPR2, HaMBL, HaWRKY53, HaOxo, and HaSTM (FIG. 6A-6F, respectively) in explants inoculated with either no inoculation (no), low inoculum (low−6×10² CFU mL⁻¹) or high inoculum (high−6×10⁸ CFU mL⁻¹) over 15 days of co-culture determined using qRT-PCR. Different letters indicate significant difference among treatments at the same time point based on Duncan's multiple range test (p<0.05).

### DETAILED DESCRIPTION

### Definitions

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein.

The terms “transgenic” and “transformation” with respect to plants refers to altering the genetic constitution of plants in the laboratory, whereby a segment of nucleic acid is introduced into plant material, which then becomes inserted in one or more of the plant chromosomes. Several methods to accomplish plant transformation have been devised. In these methods, cells of a plant or plant tissue are transformed and complete, fertile transgenic plants are recovered from that tissue. Use of the vector Agrobacterium tumefaciens is preferred because of the ability to transform plant cells.

The term “gene editing” or “genome editing” refers to a type of genetic modification in which DNA is inserted, deleted or replaced in the genome of a living organism using targeting nucleases. These nucleases create site-specific double-strand breaks (DSBs) at desired locations in the genome. The induced double-strand breaks are repaired through nonhomologous end-j oining (NHEJ) or homologous recombination (HR), resulting in targeted modifications.

The terms “crossed,” “cross,” or “crossing” as used herein refer to the fusion of gametes via pollination to produce progeny (i.e., cells, seeds, or plants). The term encompasses both sexual crosses (the pollination of one plant by another) and selfing (self-pollination, i.e., when the pollen and ovule (or microspores and megaspores) are from the same plant or genetically identical plants).

The term “dicot” as used herein refers to the subclass of angiosperm plants also knows as “dicotyledoneae” and includes reference to whole plants, plant organs (e.g., leaves, stems, roots, etc.), seeds, plant cells, and progeny of the same. Plant cell, as used herein includes, without limitation, seeds, suspension cultures, embryos, meristematic regions, callus tissue, leaves, roots, shoots, gametophytes, pollen, and microspores.

The term “monocot,” also known as “monocotyledoneae,” refers to plants whose seeds typically contain only one embryonic leaf, or cotyledon. The monocots include about 60,000 species, including orchids and grasses, as well as major grains (rice, wheat, maize, etc.) and forage grasses, sugar cane, and the bamboos. Other economically important monocot crops include various palms (Arecaceae), bananas (Musaceae), gingers and their relatives, turmeric and cardamom (Zingiberaceae), asparagus and the onions and garlic family (Amaryllidaceae). Additionally most of the horticultural bulbs, plants cultivated for their blooms, such as lilies, daffodils, irises, amaryllis, cannas, bluebells and tulips, are monocots.

The term “expression” as used herein refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The term “gene” as used herein refers to a nucleic acid fragment that expresses a functional molecule such as, but not limited to, a specific protein, including regulatory sequences preceding (5′ non-coding sequences) and following (3′ non-coding sequences) the coding sequence. The term “genome” as used herein, referring to a plant cell encompasses not only chromosomal DNA found within the nucleus, but organelle DNA found within subcellular components (e.g., mitochondria, or plastid) of the cell.

The term “heterologous” is used broadly below to indicate that the gene/sequence of nucleotides in question have been introduced into the cells in question (e.g. of a plant or an ancestor thereof) using genetic engineering, i.e. by human intervention. A heterologous gene may replace an endogenous equivalent gene, i.e. one which normally performs the same or a similar function, or the inserted sequence may be additional to the endogenous gene or other sequence. Nucleic acid sequences heterologous to a cell may be non-naturally occurring in cells of that type, variety or species.

The term “introduced” as used herein refers to providing a nucleic acid (e.g., expression construct) or protein to a cell. The term “introduced’ also includes reference to the incorporation of a nucleic acid into the genome of the cell, and includes reference to the transient provision of a nucleic acid or protein to the cell. “Introduced” further includes reference to stable or transient transformation methods. Thus, “introduced” in the context of inserting a nucleic acid fragment (e.g., a recombinant DNA construct/expression construct) into a cell, means “transformation” or “transgenic” and includes reference to the incorporation of a nucleic acid fragment into a cell where the nucleic acid fragment may be incorporated into the genome of the cell (e.g., chromosome, plastid, or mitochondrial DNA), converted into an autonomous replicon, or transiently expressed. This can take place via traditional plant transformation or genome editing.

The terms “non-naturally occurring,” “genetically modified,” or “genetically engineered” as used herein are used interchangeably and refer to the production of altered plants through the use of recombinant DNA technology.

The term “plant” refers to whole plants, plant organs, plant tissues, seeds, plant cells, seeds, and progeny of the same. Plant cells include, without limitation, cells from seeds, suspension cultures, embryos, meristematic regions, callus tissue, leaves, roots, shoots, gametophytes, pollen and microspores. Plant parts include differentiated and undifferentiated tissues including, but not limited to roots, stems, shoots, leaves, pollen, seeds, tumor tissue and various forms of cells and culture (e.g., single cells, protoplasts, embryos, and callus tissue). The plant tissue may be in a plant or in a plant organ, tissue or cell culture. The term “plant organ” refers to plant tissue or a group of tissues that constitute a morphologically and functionally distinct part of a plant. “Progeny” comprises any subsequent generation of a plant. Any commercially or scientifically valuable plant is envisaged in accordance with these embodiments of the invention.

Accordingly, plant families may comprise, but are not limited to, Alliaceae, Amaranthaceae, Amaryllidaceae, Apocynaceae, Asteraceae, Boraginaceae, Brassicaceae, Campanulaceae, Caryophyllaceae, Chenopodiaceae, Compositae, Cruciferae, Cucurbitaceae, Euphorbiaceae, Fabaceae, Gramineae, Hyacinthaceae, Labiatae, Leguminosae-Papilionoideae, Liliaceae, Linaceae, Malvaceae, Phytolaccaceae, Poaceae, Pinaceae, Rosaceae, Scrophulariaceae, Solanaceae, Tropaeolaceae, Umbelliferae and Violaceae.

Such plants include, but are not limited to, Allium cepa, Amaranthus caudatus, Amaranthus retroflexus, Antirrhinum majus, Arabidopsis thaliana, Arachis hypogaea, Artemisia sp., Avena sativa, Bellis perennis, Beta vulgaris, Brassica campestris, Brassica juncea, Calendula officinalis, Capsella bursa pastoris, Capsicum annuum, Catharanthus roseus, Chemanthus cheiri, Chenopodium album, Chenopodium amaranticolor, Chenopodium foetidum, Chenopodium quinoa, Coriandrum sativum, Cucumis melo, Cucumis sativus, Glycine max, Gomphrena globosa, Gossypium hirsutum, Gypsophila elegans, Helianthus annuus, Hyacinthus orientalis, Hyoscyamus niger, Lactuca sativa, Lathyrus odoratus, Linum usitatissimum, Lobelia erinus, Lupinus mutabilis, Lycopersicon esculentum, Lycopersicon pimpinellifolium, Melilotus albus, Momordica balsamina, Myosotis sylvatica, Narcissus pseudonarcissus, Nicandra physalodes, Nicotiana benthamiana, Nicotiana clevelandii, Nicotiana glutinosa, Nicotiana rustica, Nicotiana sylvestris, Nicotiana tabacum, Nicotiana edwardsonii, Ocimum basilicum, Petunia hybrida, Phaseolus vulgaris, Phytolacca Americana, Pisum sativum, Raphanus sativus, Ricinus communis, Rosa sericea, Salvia splendens, Senecio vulgaris, Solanum lycopersicum, Solanum melongena, Solanum nigrum, Solanum tuberosum, Solanum pimpinellifolium, Spinacia oleracea, Stellaria media, Trifolium pratense, Trifolium repens, Tropaeolum majus, Tulipa gesneriana, Vicia faba, Vicia villosa and Viola arvensis. Other plants that may be infected include Zea maize, Hordeum vulgare, Triticum aestivum, Oryza sativa and Oryza glaberrima.

A genetically modified plant includes, for example, a plant that comprises within its genome a heterologous polynucleotide introduced by a transformation step or by genome editing. The heterologous polynucleotide can be stably integrated within the genome such that the polynucleotide is passed on to successive generations. The heterologous polynucleotide may be integrated into the genome alone or as part of a recombinant DNA construct. A genetically engineered plant can also comprise more than one heterologous polynucleotide within its genome. Each heterologous polynucleotide may confer a different trait to the transgenic plant. A heterologous polynucleotide can include a sequence that originates from a foreign species, or, if from the same species, can be substantially modified from its native form. Transgenic, or genetically modified, can include any cell, cell line, callus, tissue, plant part or plant, the genotype of which has been altered by the presence of heterologous nucleic acid including those initially altered as well as those created by sexual crosses or asexual propagation from the initial transgenic. The alterations of the genome (chromosomal or extra-chromosomal) by conventional plant breeding methods, and not by the genome editing or transgenic procedures described herein, that do not result in an insertion of a foreign polynucleotide, or by naturally occurring events such as random cross-fertilization, non-recombinant viral infection, non-recombinant bacterial transformation, non-recombinant transposition, or spontaneous mutation are not intended to be regarded as genetically altered.

A fertile plant is a plant that produces viable male and female gametes and is self-fertile. Such a self-fertile plant can produce a progeny plant without the contribution from any other plant of a gamete and the genetic material contained therein.

Methods are available in the art for synthesizing plant-preferred genes. See, for example, U.S. Pat. Nos. 5,380,831, and 5,436,391, and Murray et al. (1989) Nucleic Acids Res. 17: 477-498, herein incorporated by reference. Additional sequence modifications are known to enhance gene expression in a plant host. These include, for example, elimination of: one or more sequences encoding spurious polyadenylation signals, one or more exon-intron splice site signals, one or more transposon-like repeats, and other such well-characterized sequences that may be deleterious to gene expression. The G-C content of the sequence may be adjusted to levels average for a given plant host, as calculated by reference to known genes expressed in the host plant cell. When possible, the sequence is modified to avoid one or more predicted hairpin secondary mRNA structures.

The term “polymerase chain reaction (PCR)” as used herein refers to a technique for the synthesis of specific DNA segments and consists of a series of repetitive denaturation, annealing, and extension cycles. Typically, a double-stranded DNA is heat denatured, and two primers complementary to the 3′ boundaries of the target segment are annealed to the template DNA at a lower temperature, and then a temperature insensitive polymerase extends the primer sequence at an intermediate temperature. One set of these three consecutive steps is referred to as a “cycle”.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof.

Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The term “amino acid” as used herein refers to natural and/or unnatural or synthetic amino acids, including glycine and both the D or L optical isomers, and amino acid analogs and peptidomimetics.

The term “promoter” as used herein refers to a DNA sequence capable of controlling the expression of a coding sequence or functional RNA. The promoter sequence consists of proximal and more distal upstream elements. An “enhancer” is a DNA sequence that can stimulate promoter activity, and may be an innate element of the promoter or a heterologous element inserted to enhance the level or tissue-specificity of a promoter. Promoters may be derived in their entirety from a native gene, or be composed of different elements derived from different promoters found in nature, and/or comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity. Promoters that cause a gene to be expressed in most cell types at most times are commonly referred to as “constitutive promoters”.

The term “regulatory sequences” as used herein refers to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to: promoters, translation leader sequences, 5′ untranslated sequences, 3′ untranslated sequences, introns, polyadenylation target sequences, RNA processing sites, effector binding sites, and stem-loop structures.

The terms “recombinant DNA molecule”, “recombinant construct”, “expression construct”, “construct”, and “recombinant DNA construct” are used interchangeably herein. A recombinant construct comprises an artificial combination of nucleic acid fragments, e.g., regulatory and coding sequences that may or may not be all found together in nature. For example, a construct may comprise regulatory sequences and coding sequences that are derived from different sources, or regulatory sequences and coding sequences derived from the same source, but arranged in a manner different than that found in nature.

The term “stable transformation” refers to the transfer of a recombinant DNA molecule into a genome of a host organism, including both nuclear and organellar genomes, resulting in genetically stable inheritance. In contrast, “transient transformation” refers to the transfer of a recombinant DNA molecule into the cytoplasm or nucleus of a host organism resulting in gene expression without integration or stable inheritance. Host organisms containing the transformed recombinant DNA molecule are referred to as “transgenic” organisms.

The terms “target site”, “target sequence”, “target DNA”, “target locus”, “genomic target site”, “genomic target sequence”, and “genomic target locus” are used interchangeably herein and refer to a polynucleotide sequence in the genome (including chloroplastic and mitochondrial DNA) of a plant cell at which a double-strand break is induced in the plant cell genome by a targeting endonuclease, such as Cas9. The target site can be an endogenous site in the plant genome, or alternatively, the target site can be heterologous to the plant and thereby not be naturally occurring in the genome, or the target site can be found in a heterologous genomic location compared to where it occurs in nature. The terms “endogenous target sequence” and “native target sequence” are used interchangeably herein to refer to a target sequence that is endogenous or native to the genome of a plant and is at the endogenous or native position of that target sequence in the genome of the plant.

The term “targeted mutation” as used herein refers to a mutation in a native gene that was made by altering a target sequence within the native gene using a method disclosed herein or known in the art.

The term “variant” as used herein refers to the exhibition of qualities that have a pattern that deviates from what occurs in nature.

The term “wild type” as used herein refers to the typical form of an organism, strain, gene or characteristic as it occurs in nature as distinguished from mutant or variant forms.

As used herein, “agronomically valuable trait” includes any phenotype in a plant organism that is useful or advantageous for food or feed production or food or feed products, including plant parts and plant products. Non-food agricultural products such as paper, fiber, biofuel, or multi-use crops (such as sugarcane), etc. are also included. A partial list of agronomically valuable traits includes, but is not limited to, herbicide resistance, pest resistance, vigor, development time (i.e., time to harvest), enhanced nutrient content, novel growth patterns, flavors or colors, salt, heat, drought and cold tolerance, yield, and the like. Preferably, agronomically valuable traits do not include selectable marker genes (e.g., genes encoding herbicide or antibiotic resistance used only to facilitate detection or selection of transformed cells), hormone biosynthesis genes leading to the production of a plant hormone (e.g., auxins, gibberllins, cytokinins, abscisic acid and ethylene that are used only for selection), or reporter genes (e.g., green fluorescent protein (GFP), luciferase, glucuronidase, etc.). There are numerous polynucleotides from which to choose in order to confer these and other agronomically valuable traits.

The term “Agrobacterium” as used herein refers to a soil-borne, Gram-negative, rod-shaped phytopathogenic bacterium, which has the ability to introduce part of its DNA into plant cells. Agrobacterium cells are normally rod-shaped (0.6-1.0 μm by 1.5-3.0 μm), occur singly or in pairs, without endospore, and are motile by one to six peritrichous flagella. Considerable extracellular polysaccharide slime is usually produced during growth on carbohydrate-containing media. The species of Agrobacterium, A. tumefaciens (syn. A. radiobacter), A. rhizogenes, A. rubi and A. vitis, together with Allorhizobium undicola, form a monophyletic group with all Rhizobium species, based on comparative 16S rDNA analyses. The monophyletic nature of Agrobacterium, Allorhizobium and Rhizobium and their common phenotypic generic circumscription support their amalgamation into a single genus, Rhizobium. The classification and characterization of Agrobacterium strains including differentiation of A. tumefaciens and A. rhizogenes and their various opine-type classes is a practice well known in the art (see, for example, Laboratory guide for identification of plant pathogenic bacteria, 3rd edition. (2001) N. W. Schaad, J. B. Jones, and W. Chun (eds.) ISBN 0890542635; for example, the article of Moore et al. published therein).

Pathogenic strains of Agrobacterium share a common feature; they contain at least one large plasmid, the tumor-inducing or root-inducing (Ti- and Ri-, respectively) plasmid. Virulence is determined by different regions of the plasmid including the transferred DNA (T-DNA) and the virulence (vir) genes. The virulence genes mediate transfer of T-DNA into infected plant cells, where it integrates into the plant DNA. According to the “traditional” classification, Agrobacterium includes, but are not limited to, strains of Agrobacterium tumefaciens, (which by its natural, “armed” Ti-plasmid typically causes crown gall in infected plants), and Agrobacterium rhizogenes (which by its natural, “armed” Ri-plasmid causes hairy root disease in infected host plants), Agrobacterium rubi (which in its natural, “armed” form causes cane gall on Rubus), Agrobacterium vitis (which in its natural, “armed” form causes gall formation on grape), and Agrobacterium radiobacter.

The term “Ti-plasmid” as used herein is referring to a plasmid which is replicable in Agrobacterium and is in its natural, “armed” form mediating crown gall in Agrobacterium infected plants. Infection of a plant cell with a natural, “armed” form of a Ti-plasmid of Agrobacterium generally results in the production of opines (e.g., nopaline, agropine, octopine etc.) by the infected cell. Thus, Agrobacterium strains which cause production of nopaline (e.g., strain LBA4301, C58, A208) are referred to as “nopaline-type” Agrobacterium; Agrobacterium strains which cause production of octopine (e.g., strain LBA4404, Achy, B6) are referred to as “octopine-type” Agrobacterium; and Agrobacterium strains which cause production of agropine (e.g., strain EHA105, EHA101, A281) are referred to as “agropine-type” Agrobacterium.

A “disarmed” Ti-plasmid is understood as a Ti-plasmid lacking its crown gall mediating properties but otherwise providing at least part of the functions for plant infection. Preferably, the T-DNA region of said “disarmed” plasmid is modified in a way that besides the border sequences, no functional internal Ti sequences can be transferred into the plant genome. In a preferred embodiment, when used with a binary vector system, the entire T-DNA region (including the T-DNA borders) is deleted. A “disarmed Agrobacterium” is understood as an Agrobacterium containing a disarmed Ti-plasmid.

The term “Ri-plasmid” as used herein is referring to a plasmid, which is replicable in Agrobacterium and is in its natural, “armed” form mediating hairy-root disease in Agrobacterium infected plants. Infection of a plant cell with a natural, “armed” form of an Ri-plasmid of Agrobacterium generally results in the production of opines (specific amino sugar derivatives produced in transformed plant cells such as e.g., agropine, cucumopine, octopine, mikimopine etc.) by the infected cell. Agrobacterium rhizogenes strains are traditionally distinguished into subclasses in the same way Agrobacterium tumefaciens strains are. The most common strains are agropine-type strains (e.g., characterized by the Ri-plasmid pRi-A4), mannopine-type strains (e.g., characterized by the Ri-plasmid pRi8196) and cucumopine-type strains (e.g., characterized by the Ri-plasmid pRi2659). Some other strains are of the mikimopine-type (e.g., characterized by the Ri-plasmid pRi1724).

A disarmed Ri-plasmid is understood as a Ri-plasmid lacking its hairy-root disease mediating properties but otherwise providing at least some of the functions for plant infection. Preferably, the T-DNA region of said “disarmed” Ri plasmid was modified in a way, that beside the border sequences, no functional internal Ri sequences can be transferred into the plant genome. In a preferred embodiment—when used with a binary vector system—the entire T-DNA region (including the T-DNA borders) is deleted.

### GENERAL DESCRIPTION

Disclosed herein is a method for producing genetically modified plant comprising inoculating a plant, plant cell, plant seed, or plant tissue with an Agrobacterium containing a nucleic acid of interest at an inoculum density of about 10-10,000 CFU/ml; and culturing the Agrobacterium-inoculated plant, plant cell, plant seed, or plant tissue for a period of from 7 days to 28 days, wherein said culturing results in a genetically modified plant, plant cell, plant seed, or plant tissue.

As disclosed in the Examples, using shoot producing cotyledons of sunflower, inoculation of plant tissues with low numbers of Agrobacterium (10-100 bacteria or less per tissue piece vs 1×10⁶-1×10⁸ per tissue piece as is normally practiced) was less damaging to the target tissue and allowed inoculation of the tissue with the Agrobacterium for 1 to 2 weeks, or more, leading to large increases in transformation rates and recovery of transgenic tissues, which could not previously be obtained using standard inoculation methods. The methods disclosed herein deviate from the standard protocol and have not been reported or used previously. In fact, the literature shows a standard protocol of 24-30 hours of incubation with a cell density of 10⁸ CFU/ml. (Cheng et al., In Vitro Cell. Dev. Biol.-Plant 40:31-45, 2004; Fraley et al. Proc. NatL. Acad. Sci. USA Vol. 80, pp. 4803-4807, August 1983; Clough and Bent The Plant Journal (1998) 16(6), 735-743). When bacterial density and co-culture times are evaluated and varied, one is changed while the other is kept constant. When this approach is taken, a high inoculum is used with a short co-culture period. Reduction of the inoculum density with a standard, short co-culture period reduces the number of bacteria that are able to transform the target tissue and lengthens the co-culture time with a standard bacterial inoculum density. This results in bacterial overgrowth, and damages the target tissue, leading to tissue death.

This new approach allows for alternate plant targets that were not previously considered. Since the low inoculum approach allows a long term co-culture period, targets that are either sensitive to the presence of the bacterium or would be overtaken by this bacterium, could now be used. In addition to inoculation of excised tissue, this improvement can allow seeds, seedlings, plantlets, plants, pollen, flowers and other tissues to be used, which was previously not possible or resulted in very low transformation rates.

In the disclosed methods, the inoculum density can be 10, 20, 30, 40, 50, 60, 70, 80, 90, or 100 or less CFU/ml. It can also be in the range of 100, 200, 300, 400, 500, 600, 700, 800, 900, or 1,000 CFU/ml or less. It can also be in the range of 1,000, 2,000, 3,000, 4,000, 5,000, 6,000, 7,000, 8,000, 9,000, or 10,000 CFU/ml or less.

The period of incubation of the plant, plant cell, plant tissue, or plant seed with Agrobacterium can be from a period of 7 to 28 days or more. The incubation period can be 4, 5, 6, 7, 8, 9, 10, 11, 12, 13, 14, 15, 16, 17, 18, 19, 20, 21, 22, 23, 24, 25, 26, 27, 28, 29, 30, or more days.

The inoculation is generally performed at a temperature of about 15° C. to 30° C., or about 23° C. to 28° C. The inoculation can also be done using vacuum infiltration, or by placing a small volume (1-20 μl) of dilute Agrobacterium suspension on the plant tissue.

The disclosed methods encompass the use of bacterial strains to introduce one or more genetic components into plants. Those of skill in the art would recognize the utility of Agrobacterium-mediated genetic modification methods, which are useful with both traditional methods of plant transformation, as well as with genome-editing methods, which are discussed in more detail herein.

Methods of Use

Plant/Germ Line Transformation

The methods disclosed herein can be used for the production of traditional transgenics using Agrobacterium to transform cells that contribute to germ line tissue or cells that can be regenerated into plants. The methods disclosed herein can also be used with genome editing, which is described in detail below. For transformation of germ line cells, seeds, seedlings, flowers, flower parts or plants may be inoculated with Agrobacterium or “dipped” into an Agrobacterium suspension. For in planta transformation using floral dip (Clough and Bent), Agrobacterium transforms germ line cells (egg cells), leading to the production of transgenic seed, following fertilization with pollen. In planta transformation methods are discussed in more detail below.

For Agrobacterium-mediated transformation of regenerable cells, the targeted cells are subjected to plant tissue culture, inducing the cells to regenerate into whole plants. Any suitable plant culture medium for transformation and regeneration can be used. Examples of suitable media would include but are not limited to MS-based media (Murashige and Skoog, Physiol. Plant, 15:473-497, 1962) or N6-based media (Chu et al., Scientia Sinica 18:659, 1975) supplemented with additional plant growth regulators including, but not limited to, auxins such as picloram (4-amino-3,5,6-trichloropicolinic acid), 2,4-D (2,4-dichlorophenoxyacetic acid) and dicamba (3,6-dichloroanisic acid), cytokinins such as BAP (6-benzylaminopurine) and kinetin, and gibberellins. Other media additives can include but are not limited to amino acids, macroelements, iron, microelements, vitamins and organics, carbohydrates, undefined media components such as casein hydrolysates, an appropriate gelling agent such as a form of agar, such as agarose or Gelrite if desired. Those of skill in the art are familiar with the variety of tissue culture media, which when supplemented appropriately, support plant tissue growth and development and are suitable for plant transformation and regeneration. These tissue culture media can either be purchased as a commercial preparation, or custom prepared and modified. Examples of such media would include but are not limited to Murashige and Skoog (Mursahige and Skoog, Physiol. Plant, 15:473-497, 1962), N6 (Chu et al., Scientia Sinica 18:659, 1975), Linsmaier and Skoog (Linsmaier and Skoog, Physio. Plant., 18: 100, 1965), Uchimiya and Murashige (Uchimiya and Murashige, Plant Physiol. 15:473, 1962), Gamborg's B5 media (Gamborg et al., Exp. Cell Res., 50:151, 1968), D medium (Duncan et al., Planta, 165:322-332, 1985), McCown's Woody plant media (McCown and Lloyd, HortScience 16:453, 1981), Nitsch and Nitsch (Nitsch and Nitsch, Science 163:85-87, 1969), FN medium (Finer and Nagasawa, Plant Cell Tiss & Org Cult 15:125, 1988), and Schenk and Hildebrandt (Schenk and Hildebrandt, Can. J. Bot. 50:199-204, 1972) or derivations of these media supplemented accordingly. Those of skill in the art are aware that media and media supplements such as nutrients and growth regulators for use in transformation and regeneration and other culture conditions such as light intensity during incubation, pH, and incubation temperatures that can be optimized for the particular variety of interest.

In a some embodiments, the genetic components are incorporated into a DNA composition such as a recombinant, double-stranded plasmid or vector molecule comprising at least one or more of following types of genetic components: (a) a promoter that functions in plant cells to cause the production of an RNA sequence, (b) a structural DNA sequence that causes the production of an RNA sequence that encodes a product of agronomic utility, and (c) a 3′ non-translated DNA sequence that functions in plant cells to cause the addition of polyadenylated nucleotides to the 3′ end of the RNA sequence.

Means for preparing plasmids or vectors containing the desired genetic components are well known in the art. Vectors typically consist of a number of genetic components, including but not limited to regulatory elements such as promoters, leaders, introns, and terminator sequences. Regulatory elements are also referred to as cis- or trans-regulatory elements, depending on the proximity of the element to the sequences or gene(s) they control.

Transcription of DNA into mRNA is regulated by a region of DNA usually referred to as the “promoter”. The promoter region contains a sequence of bases that signals RNA polymerase to associate with the DNA and to initiate the transcription into mRNA using one of the DNA strands as a template to make a corresponding complementary strand of RNA.

A number of promoters that are active in plant cells have been described in the literature. Such promoters would include but are not limited to the soybean (Glycine max) polyubiquitin (Gmubi) promoter (See e.g., U.S. Pat. No. 8,395,021 B2) that regulate high levels of gene expression, the nopaline synthase (NOS) and octopine synthase (OCS) promoters that are carried on tumor-inducing plasmids of Agrobacterium tumefaciens, the caulimovirus promoters such as the cauliflower mosaic virus (CaMV) 19S and 35S promoters and the figwort mosaic virus (FMV) 35S promoter, the enhanced CaMV35S promoter (e35S). All of these promoters have been used to create various types of DNA constructs that have been expressed in plants.

Promoter hybrids can also be constructed to enhance transcriptional activity, or to combine desired transcriptional activity, inducibility and tissue specificity or developmental specificity. Promoters that function in plants include but are not limited to promoters that are inducible, viral, synthetic, constitutive, and temporally regulated, spatially regulated, and spatio-temporally regulated. Other promoters that are tissue-enhanced, tissue-specific, or developmentally regulated are also known in the art and envisioned to have utility in the practice of this disclosed method.

Typically, DNA sequences located a few hundred base pairs downstream of the polyadenylation site serve to terminate transcription. The DNA sequences are referred to herein as transcription-termination regions. The regions are required for efficient polyadenylation of transcribed messenger RNA (mRNA) and are known as 3′ non-translated regions. RNA polymerase transcribes a coding DNA sequence through a site where polyadenylation occurs.

The disclosed methods can be used with any suitable plant transformation plasmid or vector containing a selectable or screenable marker and associated regulatory elements as described herein, along with one or more nucleic acids expressed in a manner sufficient to confer a particular desirable trait. Examples of suitable structural genes of agronomic interest envisioned by the disclosed method would include but are not limited to genes for insect or pest tolerance, herbicide tolerance, genes for quality improvements such as yield, nutritional enhancements, environmental or stress tolerances, or any desirable changes in plant physiology, growth, development, morphology or plant product(s). These genes of interest are discussed in more detail below.

After the construction of the vector or construct, said nucleic acid molecule, prepared as a DNA composition in vitro, is introduced into a suitable host such as E. coli and mated into another suitable host such as Agrobacterium, or directly transformed into competent Agrobacterium. These techniques are well-known to those of skill in the art and have been described for a number of plant systems including soybean, cotton, and wheat (see, for example U.S. Pat. Nos. 5,569,834, 5,159,135, and WO 97/48814 herein incorporated by reference in their entirety).

The first stage of the transformation process is the inoculation. In this stage the explants and Agrobacterium are mixed together. After inoculation, any excess Agrobacterium suspension can be removed and the Agrobacterium and target plant material are co-cultured or co-cultivated. Any number of plant tissue culture media can be used for the co-culture step. Plant tissues after inoculation with Agrobacterium can be cultured in a liquid media. The co-culture can be at a temperature of about 18° C. to 30° C., or about 23° C. to 27° C. The co-culture can be performed in the dark, light or in light-limiting conditions.

After co-culture with Agrobacterium, the explants can be placed directly onto selective media. Explants can be subcultured onto selective media in successive steps or stages, as discussed elsewhere herein. For example, the first selective media could contain a low amount of selective agent, and the next subculture could contain a higher concentration of selective agent or vice versa. The explants could also be placed directly on a fixed concentration of selective agent. Alternatively, after co-culture with Agrobacterium, the explants could be placed on media without the selective agent. Those of skill in the art are aware of the numerous modifications in selective regimes, media, and growth conditions that can be varied depending on the plant system and the selective agent. Selective markers are discussed in more detail below.

The cultures are subsequently transferred to a media suitable for the recovery of transformed plantlets. Those of skill in the art are aware of the number of methods to recover transformed plants. A variety of media and transfer requirements can be implemented and optimized for each plant system for plant transformation and recovery of transgenic plants. Consequently, such media and culture conditions disclosed in the disclosed method can be modified or substituted with nutritionally equivalent components, or similar processes for selection and recovery of transgenic events, and still fall within the scope of the disclosed methods.

The transformants produced may subsequently be analyzed to determine the presence or absence of a particular nucleic acid of interest contained on the transformation vector. Molecular analyses can include but is not limited to Southern blots (Southern, J. Mol. Biol., 98:503-517, 1975), or PCR (polymerase chain reaction) analyses, immunodiagnostic approaches, and field evaluations. These and other well-known methods can be performed to confirm the stability of the transformed plants produced by the methods disclosed. These methods are well known to those of skill in the art and have been reported (See for example, Sambrook et al., Molecular Cloning, A Laboratory Manual, 1989).

Genome Editing

The methods disclosed herein can also be used with genome editing techniques. There are a number of plant genome editing methods which make use of Agrobacterium and are known to those of skill in the art. Examples include, but are not limited to, zinc finger nucleases, TALENs, engineered meganucleases, and CRISPR-Cas9. The genome editing proteins can be used to introduce a single strand nick or a double strand break in the target gene, which is repaired and leads to a mutation in the target sequence. The genome editing proteins can also be used to introduce a desired nucleotide sequence into the target gene by homologous recombination.

The genome editing protein may be introduced into the plant cell using standard genetic engineering techniques, well known to those of skill in the art and discussed herein. The general description given above regarding promoters, expression cassettes, and markers, for example, is readily applicable to the gene editing techniques described herein. In the typical embodiment, recombinant expression cassettes comprising a polynucleotide encoding a genome editing protein of the invention can be prepared according to well-known techniques. In the case of CRISPR/Cas nuclease, the expression cassette may transcribe the guide RNA, as well.

The expression cassette can be integrated into the genome of the plant cells, in which case subsequent generations will express the genome editing proteins of the invention, unless the expression cassette is eliminated through breeding. Alternatively, the expression cassette is not integrated into the genome of the plants cell, in which case the genome editing proteins are transiently expressed in the transformed cells and are not expressed in subsequent generations.

In some embodiments, the genome editing protein itself is introduced into the plant cell. In these embodiments, the introduced genome editing protein is provided in sufficient quantity to modify the cell but does not persist after a contemplated period of time has passed or after one or more cell divisions. In such embodiments, no further steps are needed to remove or segregate away the genome editing protein and the modified cell.

In these embodiments, the genome editing protein is prepared in vitro prior to introduction to a plant cell using well known recombinant expression systems (bacterial expression, in vitro translation, yeast cells, insect cells and the like). After expression, the protein is isolated, refolded if needed, purified and optionally treated to remove any purification tags, such as a His-tag.

The genome editing protein can be expressed in Agrobacterium as a fusion protein, fused to an appropriate domain of a virulence protein that is translocated into plants (e.g., VirD2, VirE2, VirE2 and VirF). The Vir protein fused with the genome editing protein travels to the plant cell's nucleus, where the genome editing protein would produce the desired double stranded break in the genome of the cell, (see Vergunst et al. 2000 Science 290:979-82).

In Planta Transformation

In some embodiments of the invention, in planta transformation techniques (e.g., vacuum-infiltration, or floral dip procedures) are used to introduce the expression cassettes into meristematic or germline cells of a whole plant. These techniques apply to both traditional transformation as well as genome editing. Such methods provide a simple and reliable method of obtaining transformants or genome edited variants at high efficiency while avoiding the use of tissue culture, (see, e.g., Bechtold et al. 1993 C.R. Acad. Sci. 316: 1194-1199; Chung et al. 2000 Transgenic Res. 9:471-476; Clough and Bent 1998 Plant J. 16:735-743; and Desfeux et al. 2000 Plant Physiol 123:895-904). In these embodiments, seed produced by the plant comprise the expression cassettes encoding the proteins of interest. The seed can be selected based on either molecular analysis of the seedling or the ability to germinate under conditions that inhibit germination of the untransformed (or unmodified) seed.

If tissue culture is required, the transformed or genome edited cells may be regenerated into plants in accordance with techniques well known to those of skill in the art. The regenerated plants may then be grown, and crossed with the same or different plant varieties using traditional breeding techniques to produce seed, which are then selected under the appropriate conditions.

Agrobacterium

A number of wild-type and disarmed strains of Agrobacterium tumefaciens and Agrobacterium rhizogenes harboring Ti or Ri plasmids can be used for gene transfer into plants. Preferably, the Agrobacterium hosts contain disarmed Ti and Ri plasmids that do not contain the oncogenes that cause tumorigenesis or rhizogenesis, respectively, which are used as the vectors and contain the genes of interest that are subsequently introduced into plants. Preferred strains would include, but are not limited to, Agrobacterium strains LBA4404, EHA101, JTND, SBHT, GV3101, K599 and/or EHA105. The use of these strains in plant transformation has been reported and the methods are familiar to those of skill in the art. Examples include PCT/EP2005/009366, WO2015154055 A1, and EP20160166288, all of which are included by reference in their entirety.

Plants may be transformed using Agrobacterium technology, as described, for example, in U.S. Pat. Nos. 5,177,010, 5,104,310, European Patent Application No. 0131624B1, European Patent Application No. 120516, European Patent Application No. 159418B1, European Patent Application No. 176112, U.S. Pat. Nos. 5,149,645, 5,469,976, 5,464,763, 4,940,838, 4,693,976, European Patent Application No. 116718, European Patent Application No. 290799, European Patent Application No. 320500, European Patent Application No. 604662, European Patent Application No. 627752, European Patent Application No. 0267159, European Patent Application No. 0292435, U.S. Pat. Nos. 5,231,019, 5,463,174, 4,762,785, 5,004,863, and U.S. Pat. No. 5,159,135. The use of T-DNA-containing vectors for the transformation of plant cells has been intensively researched and sufficiently described in European Patent Application 120516; An et al., (1985, EMBO J. 4:277-284), Fraley et al., (1986, Crit. Rev. Plant Sci. 4:1-46), and Lee and Gelvin (2008, Plant Physiol. 146: 325-332), and is well established in the field.

Plants

The method disclosed herein are useful with whole plants, plant tissue, individual plant cells, and seeds from plants. The plant tissue or cell can originate from leaves, roots, shoots, seedlings, callus, cell suspension, cotyledon, leaf, root, egg, pollen, or microspores. The plant cell or plant tissue can be a meristem or inflorescence or parts thereof. The plant can be a monocot selected from the group consisting of maize, sorghum, triticale, barley, oats, rye, wheat, onions and rice, for example. The plant can also be a dicot selected from the group consisting of soybean, alfalfa, tobacco, brassicas, sunflower, cucurbits, potatoes, peppers and tomatoes. The plant can also be a conifer, selected from the group consisting of pine, spruce and fir. A non-exhaustive list of plants included in the embodiments herein can be found above.

Exogenous Nucleic Acids of Interest

Examples of suitable structural genes of interest include but are not limited to genes for disease, insect, or pest tolerance, herbicide tolerance, genes for quality improvements such as yield, nutritional enhancements, environmental or stress tolerances, or any desirable changes in plant physiology, growth, development, morphology or plant product(s) including starch production (U.S. Pat. Nos. 6,538,181; 6,538,179; 6,538,178; 5,750,876; 6,476,295), modified oils production (U.S. Pat. Nos. 6,444,876; 6,426,447; 6,380,462), high oil production (U.S. Pat. Nos. 6,495,739; 5,608,149; 6,483,008; 6,476,295), modified fatty acid content (U.S. Pat. Nos. 6,828,475; 6,822,141; 6,770,465; 6,706,950; 6,660,849; 6,596,538; 6,589,767; 6,537,750; 6,489,461; 6,459,018), high protein production (U.S. Pat. No. 6,380,466), fruit ripening (U.S. Pat. No. 5,512,466), enhanced animal and human nutrition (U.S. Pat. Nos. 6,723,837; 6,653,530; 6,5412,59; 5,985,605; 6,171,640), biopolymers (U.S. Pat. Nos. RE37,543; 6,228,623; 5,958,745 and U.S. Patent Publication No. US20030028917). Also environmental stress resistance (U.S. Pat. No. 6,072,103), pharmaceutical peptides and secretable peptides (U.S. Pat. Nos. 6,812,379; 6,774,283; 6,140,075; 6,080,560), improved processing traits (U.S. Pat. No. 6,476,295), improved digestibility (U.S. Pat. No. 6,531,648) low raffinose (U.S. Pat. No. 6,166,292), industrial enzyme production (U.S. Pat. No. 5,543,576), improved flavor (U.S. Pat. No. 6,011,199), nitrogen fixation (U.S. Pat. No. 5,229,114), hybrid seed production (U.S. Pat. No. 5,689,041), fiber production (U.S. Pat. Nos. 6,576,818; 6,271,443; 5,981,834; 5,869,720) and biofuel production (U.S. Pat. No. 5,998,700). Any of these or other genetic elements, methods, and transgenes may be used with the invention as will be appreciated by those of skill in the art in view of the instant disclosure.

Exemplary nucleic acids that may be introduced by the disclosed methods include, for example, DNA sequences or genes from another species, or even genes or sequences that originate with or are present in the same species, but are incorporated into recipient cells by genetic engineering methods rather than classical reproduction or breeding techniques. However, the term exogenous is also intended to refer to genes that are not normally present in the cell being transformed, or perhaps simply not present in the form, structure, etc., as found in the transforming DNA segment or gene, or genes that are normally present yet that one desires, e.g., to have over-expressed. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the plant cell, DNA from another plant, DNA from a different organism, or a DNA generated externally, such as a DNA sequence containing an antisense message of a gene, or a DNA sequence encoding a synthetic or modified version of a gene.

Alternatively, the DNA sequences of interest can affect these phenotypes by the inhibition of expression of an endogenous gene via gene silencing technologies such cosuppression, antisense, siRNA, RNAi, expression of miRNAs (natural or engineered), expression of trans-acting siRNAs, and expression of ribozymes (see e.g., U.S. Patent Application Publication 20060200878).

Numerous other possible selectable and/or screenable marker genes, regulatory elements, and other sequences of interest will be apparent to those of skill in the art. Therefore, the foregoing discussion is intended to be exemplary rather than exhaustive.

Markers

Phenotypic markers include screenable or selectable marker that includes visual markers and selectable markers, including both positive and negative selectable marker. Any phenotypic marker can be used. Specifically, a selectable or screenable marker comprises a DNA segment that allows one to identify, or select for or against a molecule or a cell that contains it, often under particular conditions. These markers can encode an activity, such as, but not limited to, production of RNA, peptide, or protein, or can provide a binding site for RNA, peptides, proteins, inorganic and organic compounds or compositions and the like.

Examples of selectable markers include, but are not limited to, DNA segments that encode products that provide resistance against otherwise toxic compounds including antibiotics, such as, spectinomycin, ampicillin, kanamycin, tetracycline, and the like; DNA segments that encode products that are otherwise lacking in the recipient cell (e.g., tRNA genes, auxotrophic markers); DNA segments that encode products which can be readily identified (e.g., phenotypic markers such as β-glucuronidase, GUS; fluorescent proteins such as green fluorescent protein (GFP), cyan (CFP), yellow (YFP), red (RFP), and cell surface proteins. Also disclosed are selectable markers that confer resistance to herbicidal compounds. Examples include nucleic acid molecules comprising a coding sequence for a modified enolpyruvylshikimate phosphate synthase (EPSPS) for glyphosate resistance, an aryloxyalkanoate dioxygenase (AAD) for 2,4-D resistance, a phosphinothricin acetyltransferase (PAT) for glufosinate resistance, as well as a dicamba mono-oxygenase (DMO) for dicamba resistance.

Progeny and Propagation Material

Disclosed herein is genetically modified plant tissue, plants or seeds modified by the methods disclosed herein. As described above, “plant tissue” refers to any tissue of a plant, in planta, or in culture. This term includes, but is not limited to, whole plants, plant cells, plant organs, plant seeds, protoplasts, callus, cell cultures and any groups of plant cells organized into structural and/or functional units. The use of this term in conjunction with, or in the absence of, any specific type of plant tissue as listed above or otherwise embraced by this definition is not limited to be exclusive of any other type of plant tissue.

The genetically engineered cells disclosed herein can be grown into plants in accordance with conventional techniques. See for example, McCormick et al. (1986) Plant Cell Reports, 5:81-84. These plants may then be grown, and either pollinated with the same transformed strain or different strains, and the resulting hybrid having the desired phenotypic characteristic identified. Two or more generations may be grown to ensure that the introduced phenotypic characteristic is stably maintained and inherited and then seeds harvested to ensure the desired phenotype or other property has been achieved. Thus, disclosed are genetically modified progeny produced by the genetically modified plants and seeds of the present invention. Also disclosed is propagation material of the genetically modified plants disclosed herein, wherein the propagation material comprises the nucleic acid of interest. Parts obtained from the regenerated plant, such as flowers, seeds, leaves, branches, fruit, and the like are covered by the invention, provided that these parts comprise cells which have been genetically modified by the methods disclosed herein.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: The Use of Low Density Inoculum and Long Term Co-Culture for Agrobacterium-Mediated Transformation of Sunflower (Helianthus annuus L.)

Materials and Methods

Plant material preparation. Seeds of sunflower (Helianthus annuus L.) RHA280 were harvested from plants grown under greenhouse conditions as previously described (Zhang and Finer 2015), and stored in sealed plastic bags at 4° C. in the dark for up to 1 yr. After pericarps were removed manually, high-quality kernels (those without developmental defects or necrotic regions) were used for transformation. Kernels were surfaced sterilized with 5% (v/v) commercial bleach (8.25% [w/v] sodium hypochlorite; Clorox, Oakland, Calif.) for 20 min, and rinsed with sterilized distilled water 8-10 times. After the embryo axis was removed by making a cut 1-2 mm from the cotyledonary node perpendicular to the longitudinal axis, the remaining cotyledons were immersed in liquid shoot induction medium (SIM). After overnight immersion, the seed coat was easily removed from the cotyledons with minimal damage to cotyledon tissues. SIM was composed of Murashige and Skoog salts (Murashige and Skoog 1962), Gamborg's B5 vitamins (Gamborg et al. 1968), 30 g L⁻¹ sucrose (MP Biomedicals, Solon, Ohio), 1.5 mg L⁻¹6-benzylaminopurine, and 0.2 mg L⁻¹ 1-naphthaleneacetic acid. The medium pH was adjusted to 5.7, and sterilized by autoclaving at 121° C. for 20 min. All medium components were obtained from Sigma-Aldrich® (St Louis, Mo.) if not otherwise specified.

Agrobacterium strain and binary vector. A. tumefaciens strain EHA105 was used for plant transformation. An expression cassette, composed of a sunflower polyubiquitin gene promoter (HaUbi, GenBank accession KX231815) cloned from RHA280, a soluble green fluorescent protein (gfp)) gene coding sequence, and a nopaline synthase (nos) terminator, was inserted into the multiple cloning site of the pCAMBIA1300 binary vector (CAMBIA, Canberra, Australia), upstream of the hygromycin phosphotransferase (hptII) gene regulated by the cauliflower mosaic virus (CaMV) 35S promoter and CaMV 35S terminator. The binary vector was introduced into EHA105 competent cells by the freeze-thaw approach (Chen et al. 1994). Bacteria were then grown on a modified YEP (yeast extract peptone) medium (pH 7.0) at 28° C. for 2 d. YEP was composed of 5 g L⁻¹ yeast extract (Thermo-Fisher Scientific, Waltham, Mass.), 10 g L⁻¹ Bacto™ peptone (Becton, Dickinson & Company, Sparks, Md.), 0.5 g L⁻¹ MgSO₄.7HO₂, 1 g L⁻¹ sucrose, solidified with 20 g L⁻¹ Bacto™ agar (Becton, Dickinson & Company), and 100 mg L⁻¹ filter-sterilized kanamycin (Thermo-Fisher Scientific). Bacterial colonies were screened for the presence of the introduced plasmid by polymerase chain reaction (PCR) using specific primers for HaUbi:gfp and virG genes (Table 1) as previously described (Benzle et al. 2015). PCR products were electrophoresed in a 1% (w/v) agarose gel stained with ethidium bromide and visualized under UV (λ=365 nm) illumination. A glycerol stock (containing 15% [v/v] sterilized glycerol) was made for overnight liquid culture from a PCR-positive colony and stored at −80° C.

Bacterial Inoculum and Plant Tissue Transformation.

Bacterial cultures were initiated from the glycerol stock, grown on solid YEP medium containing 100 mg L⁻¹ kanamycin at 25° C., and maintained for up to 1 mo. For each experiment, a new single colony was inoculated into 2 mL liquid YEP medium containing 100 mg L⁻¹ kanamycin, and incubated in the dark at 28° C. at 150 rpm. After 24 h, 500 μL of the culture was inoculated into 50 mL liquid YEP containing 100 mg L⁻¹ kanamycin, and incubated in the dark at 28° C. at 150 rpm overnight. After the optical density at λ=600 nm (OD₆₀₀) of the culture reached between 0.6 and 1.0, the bacterial culture was centrifuged at 3000×g for 10 min at 4° C. The pellet was re-suspended in inoculation medium, consisting of liquid SIM supplemented with 100 μM acetosyringone (1 M stock solution in dimethyl sulfoxide, filter-sterilized; PhytoTechnology Laboratories®, Overland Park, Kans.) and 0.02% (v/v) Silwet-77 (Lehle Seeds, Round Rock, Tex.), with OD₆₀₀ adjusted to about 0.55 (10⁸-10⁹ bacteria mL⁻¹). The Agrobacterium inoculum was incubated at 25° C. without shaking in a laminar flow hood for about 4 h before use in plant transformation experiments.

Sunflower cotyledons were inoculated with A. tumefaciens by immersing them in the bacterial suspension for 10 min, and then blotting them on filter paper. Three additional cuts were made on each cotyledon with 1-2 mm between each cut, parallel to the first cut and perpendicular to the longitudinal axis, on filter paper wetted with the bacterial suspension, producing three cotyledonary explants having two cut sides, with the distal round end discarded. Cotyledon explants were placed on SIM solidified with 0.2% (w/v) Gelrite™ (Research Products International, Mt. Prospect, Ill.), generally with a cut side close to proximal end in contact with the medium, and incubated under standard culture conditions of 25° C. with a 16 h photoperiod (40 μmol m⁻² s⁻¹) using Plant & Aquarium fluorescent lamps (Philips Lighting, Somerset, N.J.) alternating with Gro-Lux® wide spectrum fluorescent lamps (Sylvania®, Mississauga, Ontario, Canada). After 3 days of co-culture, the explants were washed with liquid SIM containing 400 mg L⁻¹ Timentin® (SmithKline Beecham Pharmaceuticals, Philadelphia, Pa.), blotted dry, and transferred to solid SIM containing 400 mg L⁻¹ Timentin and 7.5 mg L⁻¹ hygromycin B (Calbiochem, La Jolla, Calif.).

Evaluation of Inoculum Density and Co-Culture Time on Transformation.

Different inoculum densities were first evaluated using a 15-d co-culture period. Log 10 serial dilutions of a high-density A. tumefaciens suspension (OD₆₀₀≈0.55), obtained as previously described, were made using inoculation medium as the diluent. Fifteen cotyledons were immersed in 9 mL of either an undiluted A. tumefaciens suspension, or one of the 10⁻², 10⁻⁴, 10⁻⁶, 10⁻⁸, or 10⁻¹⁰ dilutions, for 10 min. The cotyledon explants were prepared and plated on solidified SIM as previously described. Cotyledon explants were co-cultured with A. tumefaciens for 15 days under standard culture conditions. To calculate the number of viable bacteria in the suspensions, 100 each of the 10⁻⁵ and 10⁻⁶ dilutions was plated on solid YEP medium containing 100 mg L⁻¹ kanamycin and incubated at 25° C. for 3 d. For each experiment, the concentration of bacteria in the 10⁻⁵ and 10⁻⁶ dilutions was used to calculate the number of colony-forming units (CFU) mL⁻¹ in each different inoculum.

To determine the effects of low and high inoculum density using short and long co-culture times, sunflower cotyledon tissues were inoculated with either undiluted A. tumefaciens suspension (referred to as high inoculum hereinafter) or the 10⁻⁶ dilution (referred to as low inoculum hereinafter), followed by either a co-culture period of 3 days (referred to as short co-culture hereinafter) or a co-culture period of 15 days (referred to as long co-culture hereinafter). The explants with short co-culture were washed with liquid SIM containing 400 mg L⁻¹ Timentin® after a 3-day co-culture period, and transferred to solid SIM containing 400 mg L⁻¹ Timentin® for further culture, while the explants with long co-culture were maintained on SIM for 15 days without interruption. Hygromycin selection was not applied in any of these four treatments. In addition, the transformation approach using high inoculum with short co-culture followed by selection with 7.5 mg L⁻¹ hygromycin (referred to as high inoculum/short co-culture plus Hyg-selection hereinafter) was included.

Observation of GFP in Plant Tissue.

GFP expression in sunflower tissues was monitored using a MZFLIII stereomicroscope (Leica, Heerbrugg, Switzerland) equipped with a GFP-2 filter set (excitation 480±40 nm; emission 510 nm) and a pE-100 light-emitting diode (Andover, Hampshire, UK) as an excitation light source. To gauge the transformation efficiency, the number of adventitious shoots expressing GFP (“GFP shoots”) for each explant was counted 15 days after inoculation. Shoots with only dispersed GFP-expressing cells that did not form a solid sector were not counted as GFP shoots. Images of explants were collected with a Nikon (Melville, N.Y.) Coolpix 990 digital camera mounted on the MZFLIII fluorescence stereomicroscope. The total number of adventitious shoots was also counted for each explant in order to measure the effects of treatments on the efficiency of shoot induction.

Detection of Agrobacterium on Explants.

Cotyledons were inoculated with low or high inoculum, followed by long co-culture on SIM as previously described. At 0, 3, 6, 9, 12, and 15 days after inoculation, seven cotyledon explants were removed from culture for each treatment, and each explant was individually placed in a 1.5 mL microfuge tube containing 100 μL liquid YEP. The cotyledon tissues were homogenized using sterilized plastic pestles (Argos Technologies, Elgin, Ill.) driven by an electric power drill. Homogenates of 100 μL for each sample were plated on YEP medium containing 100 mg L⁻¹ kanamycin. After incubation for 3 days at 25° C., the presence of Agrobacterium on explants was determined based on the growth of bacteria on YEP.

Quantitative Real-Time (qRT-PCR) for Selected Genes after Agrobacterium Inoculation.

Upregulated genes, associated with plant defense response to A. tumefaciens infection, were selected from studies of Agrobacterium inoculation of A. thaliana cell cultures (Ditt et al. 2006), inflorescence stalks (Lee et al. 2009), tobacco cell cultures (Veena et al. 2003), and wheat embryogenic calli (Zhou et al. 2013). Orthologs of the selected genes were identified in the sunflower genome by running basic local alignment search tool (BLAST) using the HeliaGene database (www.heliagene.org/HA412.v1.1.bronze.20141015) with the amino acid sequences of the selected A. thaliana genes (Table 2), and the best hit with the highest percentage of identity and the lowest expection value was chosen for each gene. Afterwards, the amino acid sequence of the best hit for each sunflower gene was used to run BLAST using The Arabidopsis Information Resource (TAIR) database to confirm that the selected sunflower gene and the best hit of A. thaliana gene belonged to the same gene family. The selected genes were HaPR1, HaPR2, HaMBL, HaWRKY 53, and HaOxo (Table 2). In addition, a sunflower ortholog of the Arabidopsis shoot meristemless (STM) gene (HaSTM) was identified and included in this study to monitor how shoot induction was influenced by different inoculation methods (Table 2). Specific primers (Table 3) for qRT-PCR were then designed using the RealTime qPCR Assay Tool (Integrated DNA Technologies, Coralville, Iowa) or the PrimerQuest® Tool (Integrated DNA Technologies).

Cotyledons were inoculated with either low or high inoculum, and explants were prepared and plated on SIM for co-culture as previously described. Explants derived from cotyledons, immersed in inoculation medium without A. tumefaciens for 10 min, were used as a non-inoculated control. Seven cotyledon explants were removed from culture at 3 h, or 3, 6, 9, 12, and 15 days after inoculation, frozen in liquid nitrogen, and stored at −80° C. RNA extraction was performed within 1 mo. Three independent experiments were conducted.

Total RNA was isolated by using the RNeasy® Plant Mini Kit (Qiagen, Hilden, Germany), and genomic DNA was removed using the on-column RNase-Free DNase Set (Qiagen) according to the manufacturer's instructions. RNA samples were screened by PCR with HaOxo primers (Table 3), which spanned an 887 bp intron, and the detection of a 1011 bp amplicon in PCR products after electrophoresis indicated the presence of genomic DNA contaminant. The samples with detectable genomic DNA contamination were further treated with the Ambion® DNA-Free Removal Kit (Thermo-Fisher Scientific) according to the manufacturer's instructions until genomic DNA was undetectable. RNA concentration was quantified using a Nanodrop® ND-1000 spectrophotometer (Thermo-Fisher Scientific), and RNA integrity was determined by gel electrophoresis. Single-strand cDNA was synthesized with a RETROscript® Reverse Transcription Kit (Thermo-Fisher Scientific) from 1-2 μg of total RNA according to the manufacturer's instructions. The products were diluted, and 5 ng cDNA was used as template for qRT-PCR.

Quantitative RT-PCRs were conducted in 20 μL reactions using iQ™ SYBR® Green Supermix (Bio-Rad, Hercules, Calif.) following the manufacturer's instructions, and the iQ™ 5 optical system (Bio-Rad) was used to measure target cDNA levels. The PCR cycling conditions were: 3 min at 95° C.; 40 cycles of 10 s at 95° C. and 30 s at 60° C.; and a melt curve profiling program with a constant increase by 0.5° C. every 30 s from 55° C. to 95° C. Each gene assay was conducted three times for each sample. The data (quantification cycle, Cq) were obtained from the iQ™ 5 optical system software (Bio-Rad), and analyzed according to the qBase relative quantification framework (Hellemans et al. 2007). Amplification efficiency of each assay was estimated based on the qRT-PCR data of a 5-log serial dilution (0.005, 0.05, 0.5, 5, 50 ng μL⁻¹) of the pooled cDNA from all samples in each independent experiment. The Cq values were converted into relative quantity and then normalized by three reference genes (Table 2) using the geNorm method (Vandesompele et al. 2002). The reference genes were HaActin, HaRPS2 and HaEFh (Table 2), and the stability of the reference genes was determined by the gene-stability measure (Vandesompele et al. 2002; Hellemans et al. 2007) (M=0.72). The means of normalized relative quantity were calculated from three independent experiments for each gene/treatment/time point.

Data Analyses.

In a transformation experiment, each treatment consisted of at least three Petri dishes, and each Petri dish contained 15 explants from five different cotyledons. The experiments were repeated at least three times using a completely randomized design. The data of transformation and shoot organogenesis were analyzed using SAS/STAT software (Version 9.4 of SAS System© 2002-2012, SAS Institute Inc., Cary, N.C.) with the GLM procedure, and means comparison was conducted using Duncan's multiple range test (α=0.05). Logarithm transformation of the normalized relative quantity data for gene expression was conducted (Rieu and Powers 2009) before data analysis, and the transformed data were analyzed by the GLM procedure for each gene/time point, followed by mean comparison using Duncan's multiple range test (α=0.05).

Results and Discussion

Transformation Efficiency was Low with High Inoculum and Short Co-Culture.

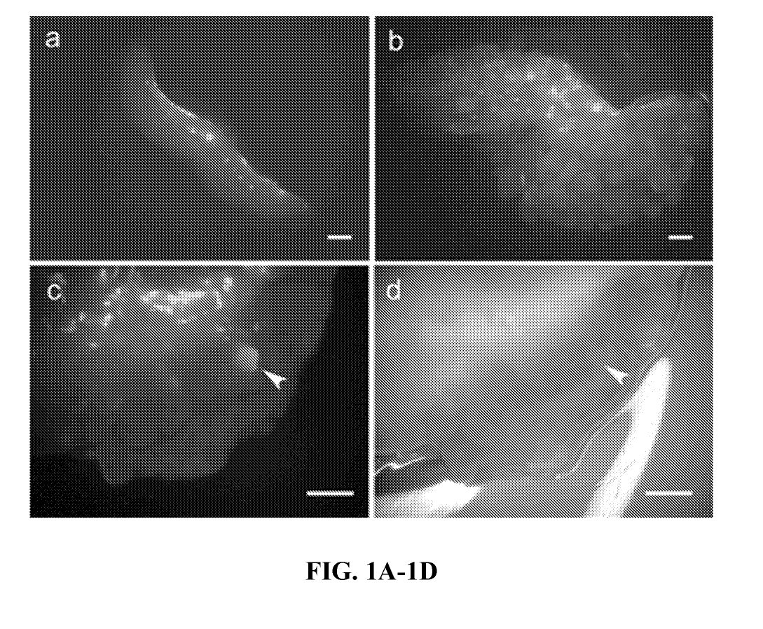

Sunflower cotyledon tissues were susceptible to Agrobacterium-mediated transformation by EHA105, and transformed cells expressing GFP were observed as early as 2 days after inoculation, following use of high inoculum (OD₆₀₀ between 0.5 and 0.6, approximately 10⁸-10⁹ CFU mL⁻¹). Despite the strong GFP expression from the HaUbi promoter, most of the GFP-expressing cells were located at the cut sides of the cotyledons, where shoot organogenesis rarely occurred (FIG. 1a). In contrast, the cells on the adaxial side of cotyledon, where adventitious shoots mostly formed, rarely showed early GFP expression using high inoculum (FIG. 1a, b). In tobacco and maize cells, transgene expression was detected within 24 hours after A. tumefaciens inoculation (Narasimhulu et al. 1996) and a 2-3 days co-culture has traditionally been used for generating transformed cells and plants (Godwin et al. 1991; Bidney et al. 1992; Hiei et al. 1994; Perl et al. 1996; Trick and Finer 1997; Bond et al. 1998; Zhao et al. 2002; Cheng et al. 2004; Ozawa 2009).

Although sunflower cotyledon explants displayed a high shoot production response, the frequency of GFP shoot production was very low with high A. tumefaciens inoculum levels and 3 days of co-culture followed by a hygromycin selection (FIG. 1b, c, d). Explant preparation generated many wounded exposed cells at the cut side of the cotyledon, and these cells apparently produced the phenolic compounds and monosaccharides that are chemotactic and inducers of A. tumefaciens virulence genes (Parke et al. 1987; Cangelosi et al. 1990). With high inoculum levels, the large numbers of bacteria apparently transformed the cells located in the wounded tissues during the early co-culture period. Although transformation of wounded sunflower tissue does not appear to be difficult, the rapidly transformed cells in the cut regions do not necessarily contribute to shoot formation. The difficulty in targeting regeneration-competent cells has been one of the major challenges in producing transgenic sunflower plants (Laparra et al. 1995).

LI/LC Increased the Frequency of GFP Shoot Production.

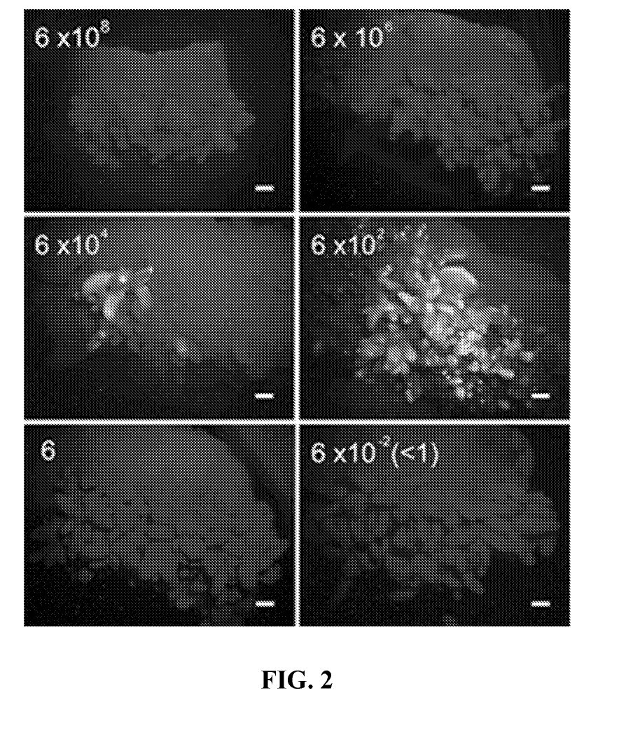

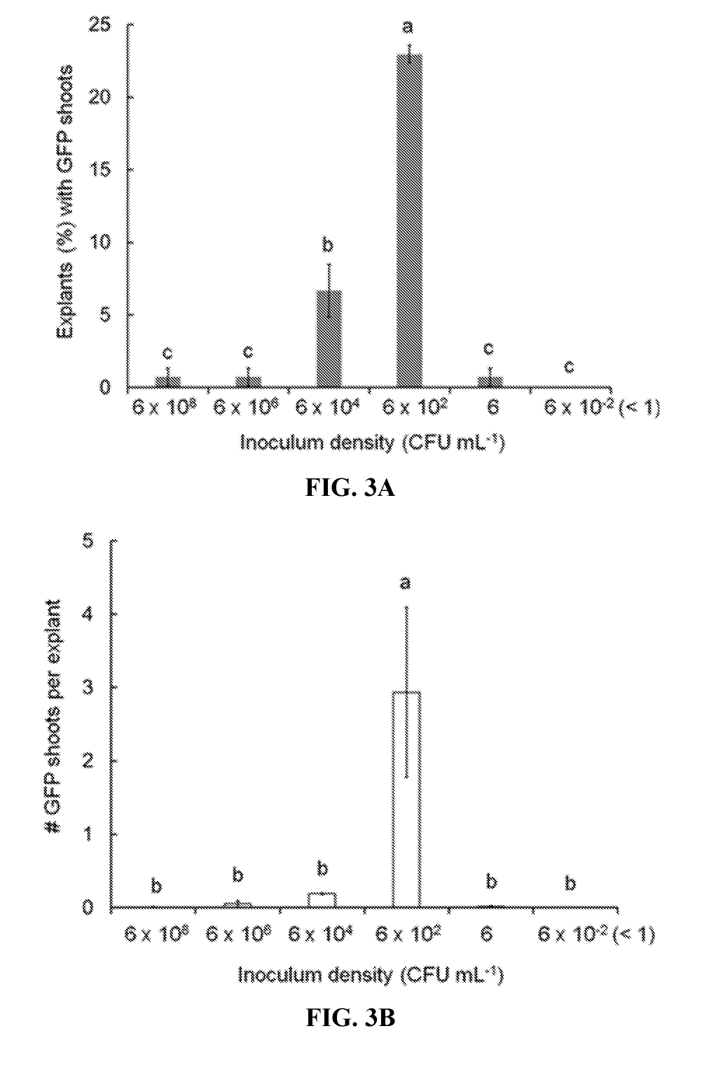

The use of low inoculum suspensions (approximately 6×10² CFU mL⁻¹) with 15-d-long co-culture resulted in the production of transformed cells at the shoot-forming regions with much higher efficiency, leading to a significantly higher number of GFP shoots than obtained using high inoculum (FIG. 2). The high inoculum (OD₆₀₀≈0.55) contained about 6×10⁸ CFU mL⁻¹, while its 10⁻², 10⁻⁴, 10⁻⁶, 10⁻⁸, and 10⁻¹⁰ dilutions contained about 6×10⁶, 6×10⁴, 6×10², 6, and 6×10⁻² CFU mL⁻¹, respectively. Unlike the high inoculum treatment, the use of diluted bacterial suspensions did not result in detectable early GFP expression in wounded tissues, probably due to the lower numbers of A. tumefaciens cells on each explant at the early time point. By the time the A. tumefaciens population increased, those early-wounded cells in the cut cotyledon may not have been as susceptible to Agrobacterium-mediated transformation, which is one explanation for why the LI/LC method led to fewer transformed cells at the cut sides than high inoculum. Regardless, the use of a low inoculum suspension with about 6×10² CFU mL⁻¹ led to a significant increase in the production of GFP shoots after a 15-d-long co-culture (FIG. 2). More than 20% of the explants exposed to this low inoculum formed GFP shoots, with about three GFP shoots per explant (FIG. 3). When bacterial density in the A. tumefaciens suspension was either higher or lower than 6×10² CFU mL⁻¹, the percentage of explants with GFP shoots and the numbers of GFP shoots per explant were both lower (FIG. 3). Interaction between inoculum density and co-culture time was observed, and low inoculum and long co-culture were both required for the increased transformation efficiency of sunflower shoots (Table 4). Neither low inoculum with short co-culture nor high inoculum with either short or long co-culture yielded any GFP shoots (Table 4). In addition, the production of transformed shoots with the LI/LC method without hygromycin selection was 30-fold higher than the traditional approach of using high inoculum/short co-culture plus Hyg-selection, where 6% explants formed GFP shoots, with 0.06 GFP shoots per explant (data not shown).

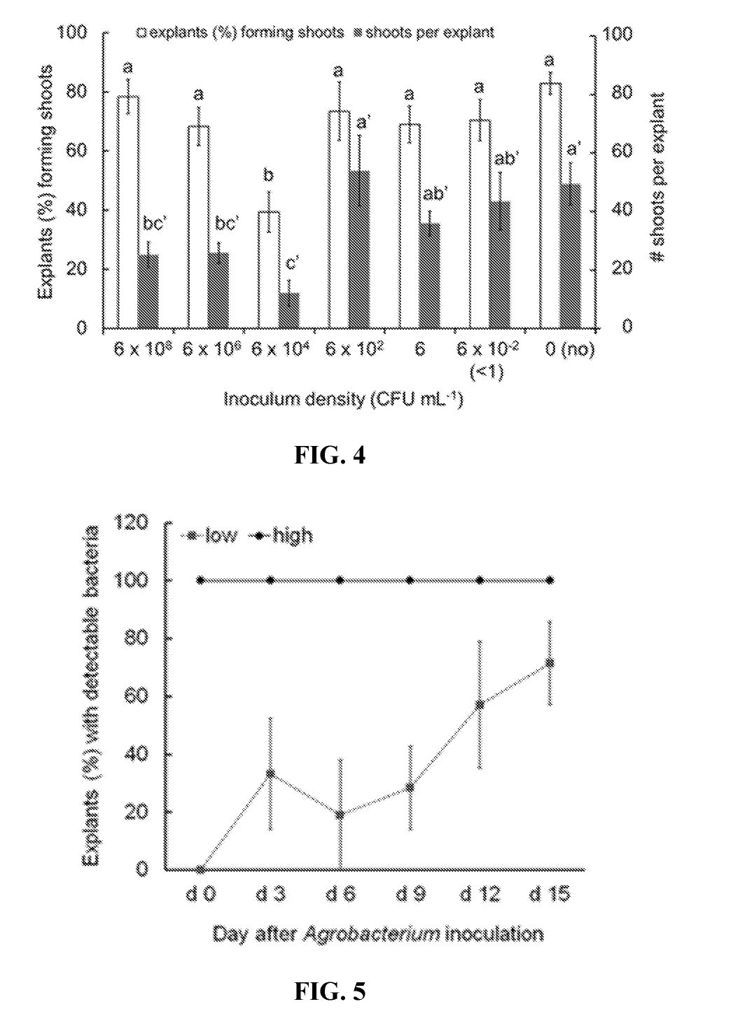

The increase of GFP shoot production by the LI/LC method could be attributable to the extended time of interaction between bacteria and plant tissues. With extended interaction, A. tumefaciens would likely have more opportunities to target and transform the rapidly growing cells that are involved in shoot formation and plant regeneration, producing more transgenic shoots. Previous attempts to increase transformation by extending co-culture period with high inoculum were unsuccessful, as plant regeneration were severely suppressed by bacterial overgrowth and no improvement of transgenic shoot production was observed (Rashid et al. 1996; Cervera et al. 1998; Sujatha et al. 2012). In the present study, the number of shoots arising from explants treated with low inoculum was similar to the number produced without inoculation, and higher than those treated with high inoculum (FIG. 4). Although alternate wounding approaches have been employed for successful transformation of sunflower tissues (Bidney et al. 1992; Grayburn and Vick 1995; Weber et al. 2003), too much wounding also reduces the regeneration response due to disruptions to organized tissues (Trick and Finer 1997). With the use of LI/LC shown here, additional wounding was not necessary for the enhanced transformation in the shoot-forming regions, making the LI/LC method valuable for tissues where wounding is undesirable. It is still possible that moderate wounding or the application of other approaches could further expand the applications of the LI/LC approach.

Low Inoculum LED to Lower Infestation Rates.

At 0 days (immediately after inoculation), A. tumefaciens cells were detected on every explant treated with high inoculum, but not on any explants treated with low inoculum (FIG. 5). Bacteria were detected in some explants 3 days after the low inoculum treatment, but the percentage of explants with detectable bacteria never reached 100%. In contrast, bacteria were detected on all the explants sampled during 15 days of co-culture after the high inoculum treatment (FIG. 5). The presence of A. tumefaciens on explants did not necessarily lead to GFP shoots. With high inoculum, GFP shoots were rarely obtained, regardless of the consistent detection of A. tumefaciens cells. Bacteria were always detected on explants producing GFP shoots. With low inoculum, bacteria were detected on explants forming GFP shoots as well as those having no GFP shoots. High inoculum could lead to visible bacterial growth on the co-culture medium within 1 week while low inoculum did not result in visible bacterial growth around explants until almost the end of the long co-culture period.

The large variation in the percentage of explants with detectable bacteria observed with the low inoculum treatment indicated some variation in the initial number of bacteria on each explant, which could explain the considerable variation of transgenic shoots arising from each explant (FIG. 3). Many explants that did not form any GFP shoots may simply not have been inoculated with a single A. tumefaciens cell or the inoculated bacteria did not survive. Using traditional inoculation methods, explants were dipped in and exposed to a bacterial suspension, but the number of bacteria that became attached to the explants could not be controlled. With an inoculum containing 10-fold more bacteria than the low inoculum, about three viable bacteria on average were detected on each explant after inoculation, suggesting that bacterial numbers on explants inoculated with low inoculum were very small, perhaps under the detection limit of the YEP plating assay. Given this situation, most of explants might not have been infected when using inocula containing less than 6×10² CFU mL⁻¹, so that diminished transformation was observed (FIGS. 2, 3).

Low Inoculum LED to Reduced Expression of Plant Defense Genes.

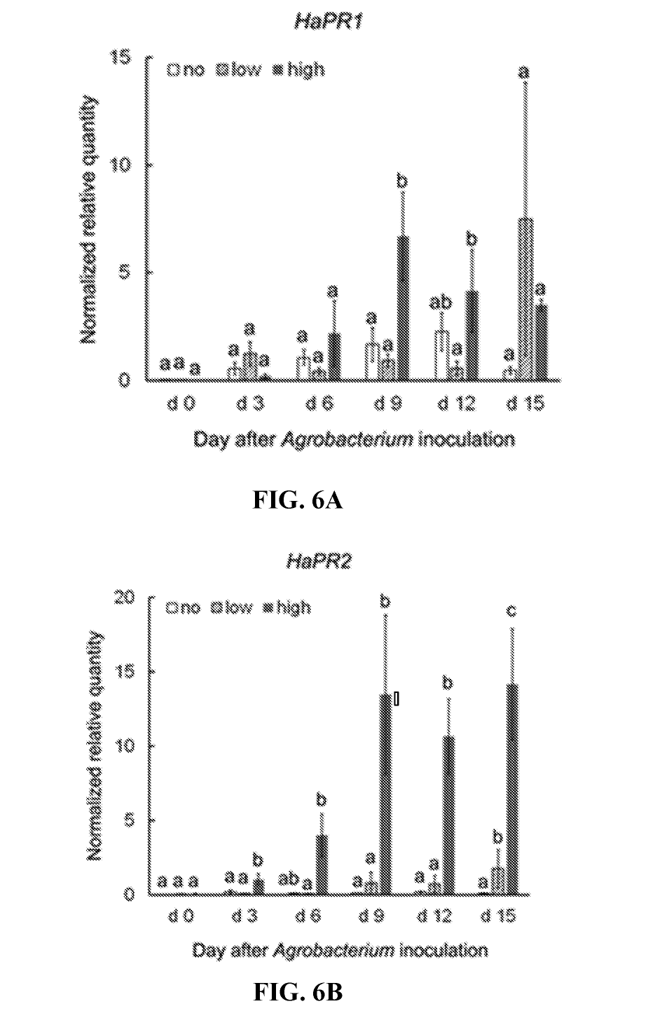

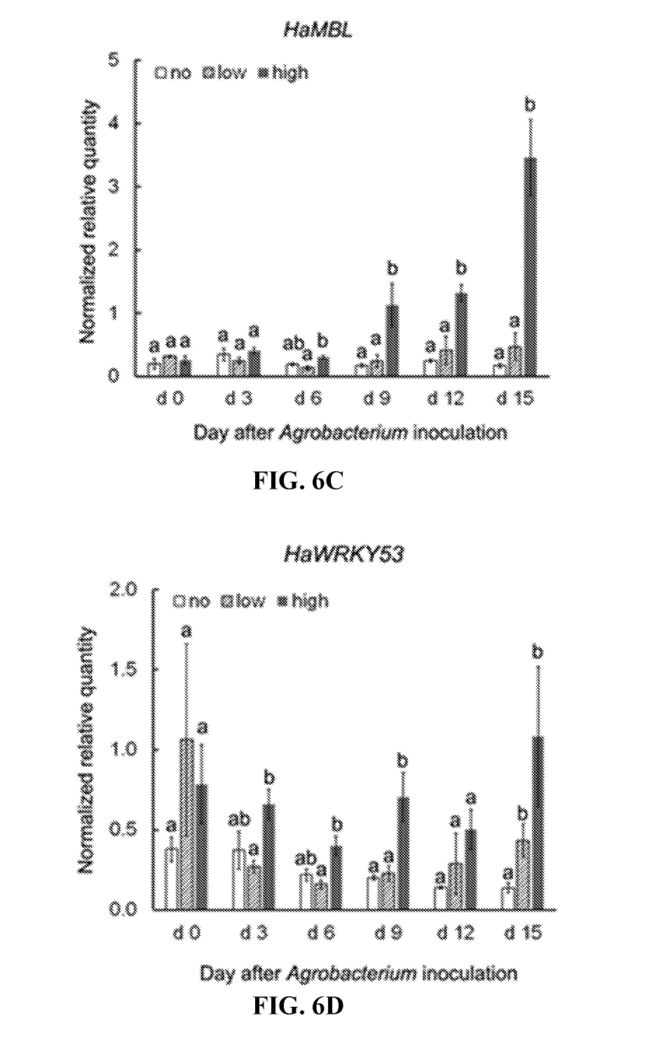

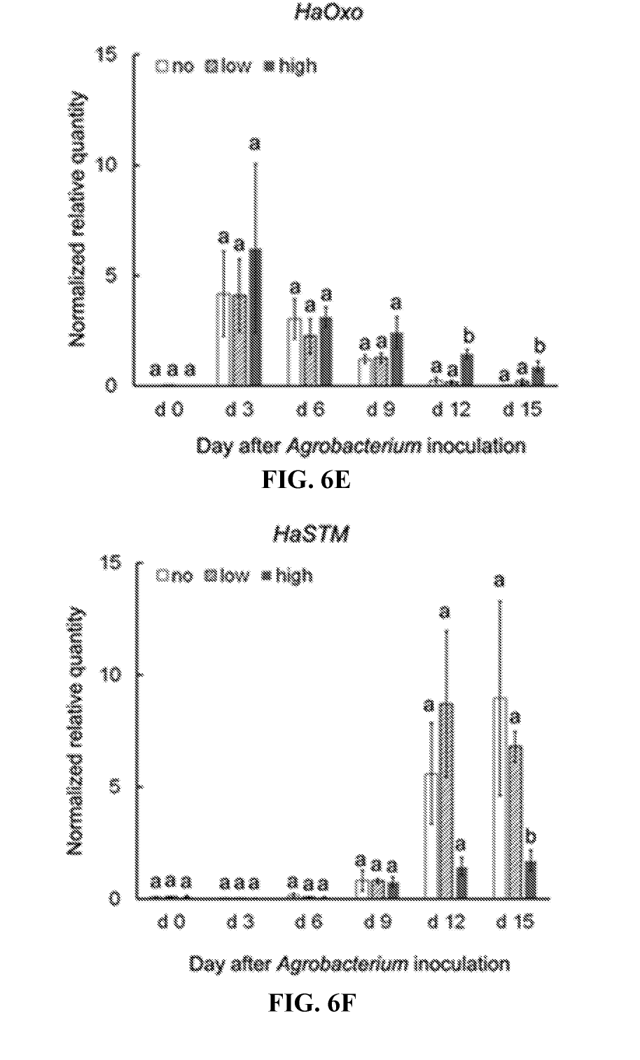

Orthologs of five sunflower genes (Table 2) were identified based on five A. thaliana genes that were upregulated after A. tumefaciens infection and contributed to defense responses (Ditt et al. 2006; Lee et al. 2009). The bidirectional BLAST analysis confirmed that the sunflower genes and the corresponding A. thaliana genes belonged to the same gene family. Differential expression of these genes was observed between the low inoculum and high inoculum treatments. Based on qRT-PCR, their expression in explants treated with low inoculum was, in general, lower than with high inoculum (FIG. 6). The lower expression levels of some key genes associated with plant defense responses in the low inoculum treatment suggested that a reduction in expression of plant defense response genes may reduce or eliminate the inhibition of Agrobacterium-mediated plant transformation (Veena et al. 2003; Zipfel et al. 2006).

A sunflower ortholog of PR protein 1 (HaPR1) was expressed at higher levels under high inoculum than low inoculum at 9 and 12 days (FIG. 6a). HaPR1 expression as 9 days after the high inoculum treatment was seven-fold higher than the low inoculum treatment, while its expression after the low inoculum treatment remained low until the end of long co-culture (FIG. 6a). After 3 days of co-culture, a sunflower ortholog of PR protein 2 (HaPR2) was expressed 20-fold higher in explants treated with high inoculum than low inoculum, and its expression continued to increase (FIG. 6b). HaPR2 expression in tissues treated with high inoculum was consistently over 10-fold higher than in tissues treated with low inoculum during co-culture from 3 to 15 days (FIG. 6b). Since the accumulation of PR proteins is associated with systematic acquired resistance (SAR) (Ward et al. 1991; Uknes et al. 1992), the higher expression of PR proteins in the high inoculum treatment indicated the activation of SAR. The induction of SAR with high inoculum may have contributed to reduced transformation rates in sunflower, as constitutive expression of PR proteins has previously been shown to confer resistance to Agrobacterium-mediated transformation in A. thaliana (Gaspar et al. 2004; Veena et al. 2003). In contrast, the use of low inoculum probably delayed or avoided the activation of SAR, leading to enhanced transformation.

A bulb-type lectin/S-locus glycoprotein/mannose-binding lectin (HaMBL) also expressed at higher levels in the explants treated with high inoculum than those treated with low inoculum from 6 to 15 days during co-culture (FIG. 6c). HaMBL expression in explants after high inoculum treatment continued to increase while its expression in explants treated with low inoculum remained almost unchanged (FIG. 6c). Since mannose-binding lectin/bulb-type lectins are potential receptors of lipopolysaccharides that are the major components of the bacterial outer membrane, and thus may serve as PAMP signals (Dow et al. 2000), induction of a mannose-binding lectin gene HaMBL by high inoculum suggests the activation of PTI that could inhibit Agrobacterium-mediated transformation. Bulb-type lectin genes have been associated with plant defense responses to bacterial pathogens in A. thaliana (Ranf et al. 2015) and pepper (Capsicum annuum, Hwang and Hwang 2011). Although a direct link between the bulb-type lectin/mannose-binding lectin and resistance to A. tumefaciens has not been clearly demonstrated, induction of expression of the HaMBL gene in this study as well as previous gene expression results from inoculating A. thaliana inflorescence stalks with A. tumefaciens (Lee et al. 2009) suggest that it functions as a receptor for some unknown PAMP signals (Zipfel et al. 2006). Accumulated PAMP signals probably activate plant defense responses that result in lower transformation efficiencies (Zipfel et al. 2006).

High inoculum led to slightly higher expression of WRKY53 than low inoculum. High expression of HaWRKY53 was observed soon after explant preparation (0 days, FIG. 6d), yet there was no significant difference among inoculation treatments. After 3 days of co-culture, the expression of HaWRKY53 started to decline in explants treated with no inoculation and low inoculum, while its expression in explants treated with high inoculum remained stable, being about two-fold higher than in explants treated with low inoculum from 3 to 9 days (FIG. 6d). HaWRKY53 expression in low inoculum increased and reached a level comparable to high inoculum at 12 and 15 days. Transcription factor WRKY53 is a marker for the early stages of senescence (Hinderhofer and Zentgraf 2001) and is also associated with stress responses (Miao et al. 2004). The relatively high expression of HaWRKY53 at 0 days could be attributable to the wounds generated during explant preparation. Higher expression of HaWRKY53 in explants treated with high inoculum from 3 to 9 days indicated that explants underwent more severe stress or more cells underwent HR than with low inoculum during this period, which could limit the transformation of the induced cells that have the potential to contribute to meristem formation and shoot formation.

An oxoglutarate/iron-dependent dioxygenase gene (HaOxo) expressed at a higher level with the high inoculum treatment than with the low inoculum treatment at 12 and 15 days during co-culture. Although no difference was observed among treatments from 0 to 9 days, its expression in the high inoculum treatment was over seven-fold higher than in the low inoculum treatment during the later stage of co-culture (FIG. 6e). Since the A. thaliana ortholog of HaOxo is associated with PCD induced by H₂O₂ (Gechev et al. 2005) and also induced by A. tumefaciens infection (Lee et al. 2009), the higher expression of HaOxo at the later stage of co-culture with high inoculum suggested that more plant cells were progressing through PCD at this time point. After exposure to large numbers of A. tumefaciens cells, plant defense was likely induced, probably leading to apoplasmic alkalization and reactive oxidative burst that could function as apoptosis signals (Mur et al. 2008) and negatively affect plant regeneration from transformed cells.

In addition, the expression of the sunflower ortholog of the Arabidopsis shoot meristemless (STM) gene (HaS™) that is related to shoot meristem formation, tended to be higher with the low inoculum treatment than the high inoculum treatment. A significant difference was observed at 15 days between the low inoculum and the high inoculum treatments (FIG. 6f). The higher expression of HaSTM in the low inoculum treatment suggested higher shoot meristem activity in the explants treated with low inoculum than high inoculum. Higher shoot induction responses were observed with the low inoculum treatment compared to the high inoculum treatment (FIG. 2). A high shoot induction response likely contributed to a higher production of transformed shoots, when low inoculum as used with long co-culture.

### CONCLUSIONS

A novel Agrobacterium-mediated transformation procedure was developed for sunflower, using low inoculum at about 6×10² CFU mL⁻¹ with a long co-culture period of 15 d. With high inoculum and short (2-3 days) co-culture, the single cells that were transformed were located in freshly cut tissues. These transformed cells apparently did not contribute to later plant regeneration. In contrast, use of low inoculum with a long co-culture period increased the chances of transforming cells that contributed to meristem formation. The use of low inoculum allowed an extension of co-culture time, increasing the opportunity for A. tumefaciens to interact with appropriate target cells. As opposed to high inoculum, use of low inoculum levels did not lead to suppression of plant regeneration or activation of defense responses. The use of LI/LC may be more similar to the infestation of plants by Agrobacterium sp. in nature, where relatively low amounts of bacteria are found in the soil (Benzle et al. 2015).

Tables

TABLE 1
Primer sequences for amplification of the HaUbi
promoter, gfp, and virG

| PRIMER | |
|---|---|
| NAME | PRIMER SEQUENCES |
| HaUbiF | CGCAGTAGTTTGAAAGTAACCC (SEQ ID NO: 1) |
| HaUbiR | CAAACAGATTAATACCCTAAGC (SEQ ID NO: 2) |
| gfpR | CCGTAGGTGGCATCGC (SEQ ID NO: 3) |
| VirGF | ATCTYAATTTRGGKCGYGAAGA (SEQ ID NO: 4) |
| VirGR | CACRTCMGCGTCRAAGAAATA (SEQ ID NO: 5) |

TABLE 2
Sunflower orthologs studied by quantitative RT-PCR analysis and the three reference genes

| SUNFLOWER | HELIAGENE DATABASE | | TAIR: Arabidopsis |
|---|---|---|---|
| ORTHOLOGS | GENE IDENTIFIERS | PUTATIVE FUNCTION | GENE IDENTIFIERS |
| HaPR1 | Ha412v1r1_12g021430 | Pathogenesis-related protein 1/ | At2g19970 |
| | | Allergen V5/Tpx-1-related | |
| HaPR2 | Ha412v1r1_14g008310 | Pathogenesis-related protein 2/ | At2g43610 |
| | | Glycoside hydrolase | |
| HaMBL | Ha412v1r1_11g021150 | Mannose-binding lectin | At1g78850 |
| | | Bulb-type lectin/S-locus | |
| | | glycoprotein | |
| HaWRKY53 | Ha412v1r1_00g070960 | WRKY53 transcription factor | At4g23810 |
| HaOxo | Ha412v1r1_13g029660 | Oxoglutarate/ | At3g13610 |
| | | iron-dependent dioxygenase | |
| HaSTM | Ha412v1r1_15g010080 | Homeodomain transcription | At1g62360 |
| | | factor | |
| | | Shoot meristemless/KNOX | |
| | | family | |

TABLE 3
Primer sequences for quantitative real-time PCR

| | | | EXPECTED |
|---|---|---|---|
| GENE | | | AMPLICON |
| NAMES | FORWARD PRIMER | REVERSE PRIMER | SIZE |
| HaPR1 | GGTGGACCTTATGGTGA | CACGTATTGGTAGTGTGG | 113 bp |
| | GAAC (SEQ ID NO: 6) | TCATA (SEQ ID NO: 7) | |
| HaPR2 | TTGGTTCCGCTAGTTCAG | GGATATTGGGTGTTAAGT | 148 bp |
| | AAG (SEQ ID NO: 8) | TCGTC (SEQ ID NO: 9) | |
| HaMBL | CAAACCCTACATCATCGT | CTAAGGTTTCCGTCTATC | 83 bp |
| | CAAATC (SEQ ID NO: 10) | CCTAATC (SEQ ID NO: 11) | |
| HaWRKY53 | CGATGACGGTTATAGTTG | TCGTCTGTTCTCTGCACT | 135 bp |
| | GAGG (SEQ ID NO: 12) | TG (SEQ ID NO: 13) | |
| HaOxo | GAGTGGAAGGATTATCT | ACCTCTTGACAACCGTTT | 124 bp |
| | CAGCC (SEQ ID NO: 14) | CAG (SEQ ID NO: 15) | |
| HaSTM | GTCTCATCCTCATTACCC | CGGAGCGACTGGACATT | 149 bp |
| | TCG (SEQ ID NO: 16) | G (SEQ ID NO: 17) | |
| HaActin | CCCAGTTCTCTTAGGCAC | GCCCTCAAATATGTCCCT | 141 bp |
| | AAC (SEQ ID NO: 18) | ACG (SEQ ID NO: 19) | |
| HaRps2 | GATGGTTTTGCAGATGCG | TCCTCTGGTTCCCTGTAG | 87 bp |
| | AG (SEQ ID NO: 20) | AAG (SEQ ID NO: 21) | |
| HaEFh | GCTTTCAGCCTCTTCGAC | CCATCAGCATCCACCTCA | 134 bp |
| | AAG (SEQ ID NO: 22) | TTG (SEQ ID NO: 23) | |

TABLE 4
The effect of inoculum density and co-culture
time on sunflower transformation

| | Percentage of explants with GFP shoots | |
|---|---|---|
| | (Numbers of GFP shoots per explant) | |
| | Agrobacterium inoculum density (CFU mL⁻¹) | |
| Co-culture time | 6 × 10⁸ | 6 × 10² |
| 3 d | 0.0 ± 0.0 (0.0 ± 0.0) a | 0.0 ± 0.0 (0.0 ± 0.0) a |
| 15 d | 0.0 ± 0.0 (0.0 ± 0.0) a | 23.7 ± 2.3 (1.8 ± 0.7) b |

Data represent mean ± standard error based on 3 replicates, each replicate containing 45 explants.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

### REFERENCES

Alt-Mörbe J, Kuhlmann H, Schröder J (1989) Differences in induction of Ti plasmid virulence genes virG and virD, and continued control of virD expression by four external factors. Mol Plant Microbe Interact 2:301-308
Benzle K A, Finer K R, Marty D, McHale L K, Goodner B W, Taylor C G, Finer J J (2015) Isolation and characterization of novel Agrobacterium strains for soybean and sunflower transformation. Plant Cell Tissue Organ Cult 121:71-81
Bevan M W, Flavell R B, Chilton M (1983) A chimaeric antibiotic resistance gene as a selectable marker for plant cell transformation. Nature 304:184-187
Bidney D, Scelonge C, Martich J, Burrus M, Sims L, Huffman G (1992) Microprojectile bombardment of plant tissues increases transformation frequency by Agrobacterium tumefaciens. Plant Mol Biol 18:301-313
Blanc G, Baptiste C, Oliver G, Martin F, Montoro P (2006) Efficient Agrobacterium tumefaciens-mediated transformation of embryogenic calli and regeneration of Hevea brasiliensis Müll Arg. plants. Plant Cell Rep 24:724-733
Bond J E, Roose M L (1998) Agrobacterium-mediated transformation of the commercially important citrus cultivar Washington navel orange. Plant Cell Rep 18:229-234
Cangelosi G A, Ankenbauer R G, Nester E W (1990) Sugars induce the Agrobacterium virulence genes through a periplasmic binding protein and a transmembrane signal protein. Proc Natl Acad Sci USA 87:6708-6712
Cervera M, Pina J, Juarez J, Navarro L, Pena L (1998) Agrobacterium-mediated transformation of citrange: factors affecting transformation and regeneration. Plant Cell Rep 18:271-278
Chen H, Nelson R S, Sherwood J L (1994) Enhanced recovery of transformants of Agrobacterium tumefaciens after freeze-thaw transformation and drug selection. Biotechniques 16:664-668
Cheng M, Hu T, Layton J, Liu C N, Fry J E (2003) Desiccation of plant tissues post-Agrobacterium infection enhances T-DNA delivery and increases stable transformation efficiency in wheat. In Vitro Cell Dev Biol—Plant 39:595-604
Cheng M, Lowe B A, Spencer T M, Ye X, Armstrong C L (2004) Factors influencing Agrobacterium-mediated transformation of monocotyledonous species. In Vitro Cell Dev Biol-Plant 40:31-45
Clough S J, Bent A F (1998) Floral dip: a simplified method for Agrobacterium-mediated transformation of Arabidopsis thaliana. Plant J 16:735-743
Coll N S, Epple P, Dangl, J L (2011) Programmed cell death in the plant immune system. Cell Death Differ 18:1247-1256
de Ropp R S (1947) The growth-promoting and tumefacient factors of bacteria-free crown-gall tumor tissue. Am J Bot 34:248-261
Ditt R F, Kerr K F, de Figueiredo P, Delrow J, Comai L, Nester E W (2006) The Arabidopsis thaliana transcriptome in response to Agrobacterium tumefaciens. Mol Plant Microbe Interact 19:665-681
Dow M, Newman M A, von Roepenack E (2000) The induction and modulation of plant defense responses by bacterial lipopolysaccharides. Annu Rev Phytopathol 38:241-261
Everett N P, Robinson K E P, Mascarenhas D (1987) Genetic engineering of sunflower (Helianthus annuus L.) Nat Biotechnol 5:1201-1204
Fillatti J J, Sellmer J, McCown B, Haissig B, Comai L (1987) Agrobacterium mediated transformation and regeneration of Populus. Mol Gen Genet 206:192-199
Fraley R T, Rogers S G, Horsch R B, Sanders P R, Flick J S, Adams S P, Bittner M L, Brand L A, Fink C L, Fry J S, Galluppi G R, Goldberg S B, Hoffmann N L, Woo S C (1983) Expression of bacterial genes in plant cells. Proc Natl Acad Sci USA 80:4803-4807
Frame B R, Shou H, Chikwamba R K, Zhang Z, Xiang C, Fonger™, Pegg S E K, Li B, Nettleton D S, Pei D, Wang K (2002) Agrobacterium tumefaciens-mediated transformation of maize embryos using a standard binary vector system. Plant Physiol 129:13-22
Gamborg O L, Miller R, Ojima K (1968) Nutrient requirements of suspension cultures of soybean root cells. Exp Cell Res 50:151-158
Gaspar Y M, Nam J, Schultz C J, Lee L Y, Gilson P R, Gelvin S B, Bacic A (2004) Characterization of the Arabidopsis lysine-rich arabinogalactan-protein AtAGP17 mutant (rat1) that results in a decreased efficiency of Agrobacterium transformation. Plant Physiol 135:2162-2171
Gechev T, Minkov I, Hille J (2005) Hydrogen peroxide induced cell death in Arabidopsis: transcriptional and mutant analysis reveals a role of an oxoglutarate dependent dioxygenase gene in the cell death process. IUBMB Life 57:181-188
Gelvin S B (2003) Agrobacterium-mediated plant transformation: the biology behind the “gene-jockeying” tool. Microbiol Mol Biol Rev 67:16-37
Gelvin S B (2012) Traversing the cell: Agrobacterium T-DNA's journey to the host genome. Front Plant Sci 3:52
Godwin I, Todd G, Ford-Lloyd B, Newbury H J (1991) The effects of acetosyringone and pH on Agrobacterium-mediated transformation vary according to plant species. Plant Cell Rep 9:671-675
Grayburn W S, Vick B A (1995) Transformation of sunflower (Helianthus annuus L.) following wounding with glass beads. Plant Cell Rep 14:285-289
Hansen G, Das A, Chilton M D (1994) Constitutive expression of the virulence genes improves the efficiency of plant transformation by Agrobacterium. Proc Natl Acad Sci USA 91:7603-7607
Hansen G (2000) Evidence for Agrobacterium-induced apoptosis in maize cells. Mol Plant Microbe Interact 13:649-657
Hellemans J, Mortier G, De Paepe A, Speleman F, Vandesompele J (2007) qBase relative quantification framework and software for management and automated analysis of real-time quantitative PCR data. Genome Biol 8:R19
Herrera-Estrella L, Block M D, Messens E, Hernalsteens J P, Montagu M V, Schell J (1983) Chimeric genes as dominant selectable markers in plant cells. EMBO J 2:987-995
Hiei Y, Ohta S, Komari T, Kumashiro T (1994) Efficient transformation of rice (Oryza sativa L.) mediated by Agrobacterium and sequence analysis of the boundaries of the T-DNA. Plant J 6:271-282
Hinderhofer K, Zentgraf U (2001) Identification of a transcription factor specifically expressed at the onset of leaf senescence. Planta 213:469-473
Hood E E, Gelvin S B, Melchers L S, Hoekema A (1993) New Agrobacterium helper plasmids for gene transfer to plants. Transgenic Res 2:208-218
Hwang I S, Hwang B K (2011) The pepper mannose-binding lectin gene CaMBL1 is required to regulate cell death and defense responses to microbial pathogens. Plant Physiol 155:447-463
Jones J D, Dangl J L (2006) The plant immune system. Nature 444:323-329
Lamb C, Dixon R A (1997) The oxidative burst in plant disease resistance. Annu Rev Plant Physiol Plant Mol Biol 48:251-275
Laparra H, Burrus M, Hunold R, Damm B, Bravo-Angel A M, Bronner R, Hahne G (1995) Expression of foreign genes in sunflower (Helianthus annuus L.)—evaluation of three gene transfer methods. Euphytica 85:63-74
Lee C W, Efetova M, Engelmann J C, Kramell R, Wasternack C, Ludwig-Müller J, Hedrich R, Deeken R (2009) Agrobacterium tumefaciens promotes tumor induction by modulating pathogen defense in Arabidopsis thaliana. Plant Cell 21:2948-2962
Leeman M, van Pelt J A, den Ouden F M, Heinsbroek M, Bakker PAHM, Schippers B (1995) Induction of systemic resistance by Pseudomonas fluorescens in radish cultivars differing in susceptibility to fusarium wilt, using a novel bioassay. Eur J Plant Pathol 101:655-664
Miao Y, Laun T, Zimmermann P, Zentgraf U (2004) Targets of the WRKY53 transcription factor and its role during leaf senescence in Arabidopsis. Plant Mol Biol 55:853-867
Mur L A, Kenton P, Lloyd A J, Ougham H, Prats E (2008) The hypersensitive response; the centenary is upon us but how much do we know? J Exp Bot 59:501-520
Murai N, Kemp J D, Sutton D W, Murray M G, Slightom J L, Merlo D J, Reichert N A, Sengupta-Gopalan C, Stock C A, Barker R F, Hall T C (1983) Phaseolin gene from bean is expressed after transfer to sunflower via tumor-inducing plasmid vectors. Science 222:476-482
Murashige T, Skoog F (1962) A revised medium for rapid growth and bio assays with tobacco tissue cultures. Physiol Plant 15:473-497
Narasimhulu S B, Deng X B, Sarria R, Gelvin S B (1996) Early transcription of Agrobacterium T-DNA genes in tobacco and maize. Plant Cell 8:873-886
Olhoft P, Somers D (2001) L-cysteine increases Agrobacterium-mediated T-DNA delivery into soybean cotyledonary-node cells. Plant Cell Rep 20:706-711
Ozawa K (2009) Establishment of a high efficiency Agrobacterium-mediated transformation system of rice (Oryza sativa L.) Plant Sci 176:522-527
Parke D, Ornston L N, Nester E W (1987) Chemotaxis to plant phenolic inducers of virulence genes is constitutively expressed in the absence of the Ti plasmid in Agrobacterium tumefaciens. J Bacteriol 169:5336-5338
Perl A, Lotan O, Abu-Abied M, Holland D (1996) Establishment of an Agrobacterium-mediated transformation system for grape (Vitis vinifera L.): the role of antioxidants during grape-Agrobacterium interactions. Nat Biotechnol 14:624-628
Power C J (1987) Organogenesis from Helianthus annuus inbreds and hybrids from the cotyledons of zygotic embryos. Am J Bot 74:497-503
Raaijmakers J M, Leeman M, van Oorschot M M, van der Sluis I, Schippers B, Bakker PAHM (1995) Dose-response relationships in biological control of fusarium wilt of radish by Pseudomonas spp. Phytopathology 85:1075-1080
Ranf S, Gisch N, Schäffer M, Illig T, Westphal L, Knirel Y A, Sánchez-Carballo P M, Zahringer U, Hückelhoven R, Lee J, Scheel D (2015) A lectin S-domain receptor kinase mediates lipopolysaccharide sensing in Arabidopsis thaliana. Nat Immunol 16:426-433
Rashid H, Yokoi S, Toriyama K, Hinata K (1996) Transgenic plant production mediated by Agrobacterium in indica rice. Plant Cell Rep 15:727-730
Rieu I, Powers S J (2009) Real-time quantitative RT-PCR: design, calculations, and statistics. Plant Cell 21:1031-1033
Santarém E R, Trick H N, Essig J S, Finer J J (1998) Sonication-assisted Agrobacterium-mediated transformation of soybean immature cotyledons: optimization of transient expression. Plant Cell Rep 17:752-759
Sujatha M, Vijay S, Vasavi S, Reddy P V, Rao S C (2012) Agrobacterium-mediated transformation of cotyledons of mature seeds of multiple genotypes of sunflower (Helianthus annuus L.). Plant Cell Tissue Organ Cult 110:275-287
Trick H N, Finer J J (1997) SAAT: Sonication-assisted Agrobacterium-mediated transformation. Transgenic Res 6:329-336
Uknes S, Mauch-Mani B, Moyer M, Potter S, Williams S, Dincher S, Chandler D, Slusarenko A, Ward E, Ryals J (1992) Acquired resistance in Arabidopsis. Plant Cell 4:645-656
Vandesompele J, De Preter K, Pattyn F, Poppe B, Van Roy N, De Paepe A, Speleman F (2002) Accurate normalization of real-time quantitative RT-PCR data by geometric averaging of multiple internal control genes. Genome Biot 3:1-12
Veena, Jiang H, Doerge R W, Gelvin S B (2003) Transfer of T-DNA and Vir proteins to plant cells by Agrobacterium tumefaciens induces expression of host genes involved in mediating transformation and suppresses host defense gene expression. Plant J 35:219-236
Ward E R, Uknes S J, Williams S C, Dincher S S, Wiederhold D L, Alexander D C, Ahl-Goy P, Métraux J P, Ryals J A (1991) Coordinate gene activity in response to agents that induce systemic acquired resistance. Plant Cell 3:1085-1094
Weber S, Friedt W, Landes N, Molinier J, Himber C, Rousselin P, Hahne G, Horn R (2003) Improved Agrobacterium-mediated transformation of sunflower (Helianthus annuus L.): assessment of macerating enzymes and sonication. Plant Cell Rep 21:475-482
Zhao Z, Gu W, Cai T, Tagliani L, Hondred D, Bond D, Schroeder S, Rudert M, Pierce D (2002) High throughput genetic transformation mediated by Agrobacterium tumefaciens in maize. Mol Breed 8:323-333
Zipfel C, Kunze G, Chinchilla D, Caniard A, Jones J D, Boller T, Felix G (2006) Perception of the bacterial PAMP EF-Tu by the receptor EFR restricts Agrobacterium-mediated transformation. Cell 125:749-760
Zhang Z, Finer J J (2015) Sunflower (Helianthus annuus L.) organogenesis from primary leaves of young seedlings preconditioned by cytokinin. Plant Cell Tissue Organ Cult 123:645-655
Zhou X, Wang K, Lv D, Wu C, Li J, Zhao P, Ye X (2013) Global Analysis of differentially expressed genes and proteins in the wheat callus infected by Agrobacterium tumefaciens. PloS One 8:e79390

1. A method for producing a genetically modified plant comprising
(a) inoculating a plant, plant cell, plant seed, or plant tissue with an Agrobacterium containing a nucleic acid of interest at an inoculum density of about 10-10,000 CFU/ml;
(b) culturing the Agrobacterium-inoculated plant, plant cell, plant seed, or plant tissue for a period of from 7 days to 28 days, wherein said culturing results in a genetically modified plant, plant cell, plant seed, or plant tissue.

2. The method of claim 1, wherein the inoculum density is about 10-1,000 CFU/ml.

3. The method of claim 1, wherein the inoculum density is about 10-100 CFU/ml.

4. The method of claim 1, wherein culturing occurs from a period of 10 days to 21 days.

5. The method of claim 1 wherein the plant is a monocot selected from the group consisting of maize, sorghum, triticale, barley, oats, rye, wheat, onions and rice.

6. The method of claim 1 wherein the plant is a dicot selected from the group consisting of soybean, alfalfa, tobacco, brassicas, sunflower, cucurbits, potatoes, peppers and tomatoes.

7. The method of claim 1, wherein the plant is a conifer selected from the group consisting of pine, spruce and fir.

8. The method of claim 1, wherein the plant is a whole plant.

9. The method of claim 1, wherein the plant tissue or cell originates from leaves, roots, shoots, seedlings, callus, cell suspension, cotyledon, leaf, root, egg, pollen, or microspores.

10. The method of claim 1, wherein the plant cell or plant tissue is a meristem or inflorescence.

11. The method of claim 1, wherein a genetically modified plant is recovered from the plant, plant cell, or plant tissue from step b).

12. The method of claim 1, wherein the Agrobacterium is genetically modified.

13. The method of claim 1, wherein an expression vector comprises the nucleic acid of interest.

14. The method of claim 1, wherein the nucleic acid of interest comprises a selectable marker.

15. The method of claim 14, wherein the selectable marker is antibiotic resistance.

16. The method of claim 14, wherein the selectable marker is herbicide resistance.

17. The method of claim 1, wherein the nucleic acid of interest comprises a screenable marker.

18. The method of claim 17, wherein the screenable marker is β-glucuronidase (GUS), luciferase or green fluorescent protein (GFP).

19. The method of claim 13, wherein the expression vector comprises a gene editing system.

20. (canceled)

21. The method of claim 19, wherein the gene editing system comprises TALENs, zinc-finger nucleases, or a CRISPR/Cas9 system.

22-30. (canceled)
